(19) United States
(12) Patent Application Publication
(10) Pub. No.: US 2009/0074750 A1
(43) Pub. Date: Mar. 19, 2009

(54) REGULATOR OF BASAL CELLULAR CALCIUM CONCENTRATION AND METHODS OF USE

(76) Inventors: Onn Brandman, Palo Alto, CA (US); Tobias Meyer, Menlo Park, CA (US)

Correspondence Address: Stanford University Office of Technology Licensing;Bozicevic, Field & Francis LLP, 1900 University Avenue, Suite 200, East Palo Alto, CA, 94303, US

(21) Appl. No.: 12/210,985

(22) Filed: Sep. 15, 2008

Related U.S. Application Data
(60) Provisional application No. 60/973,402, filed on Sep. 18, 2007.

(51) Int. Cl. A61K 39/395; C12Q 1/02; C12Q 1/68; C12N 5/06

(52) U.S. Cl. 424/130.1; 435/29; 435/6; 435/375

(57) ABSTRACT

The invention features methods and compositions for determining states of basal intracellular calcium levels in a eukaryotic cell. Also provided are methods for identifying an agent (e.g., a gene product or small molecule compound) that modulates basal intracellular calcium levels (e.g., by modulating STIM-2 activity), as well as kits and systems for practicing the subject methods.

### GOVERNMENT RIGHTS

This invention was made with government support under federal grant no. MH64801 awarded by National Institutes of Health. The United States Government has certain rights in this invention.

### BACKGROUND OF THE INVENTION

Ionic calcium (Ca²⁺) is a ubiquitous second messenger that regulates secretion, contraction, gene expression and other cell functions. In unstimulated cells, the basal cytosolic concentration of Ca²⁺ is kept constant at a concentration that is four orders of magnitude below the Ca²⁺ concentration outside of the cell and about three orders of magnitude below the Ca²⁺ concentration in the endoplasmic reticulum (ER) (Berridge et al., 2003). Receptor stimuli typically increase Ca²⁺ concentration up to ten-fold from basal by opening Ca²⁺ channels in the plasma membrane (PM) or ER membrane (Gallo et al., 2006). These Ca²⁺ signals are generated by a dynamic system that relies on Ca²⁺ channels and pumps in the PM as well as on channels and pumps in ER Ca²⁺ stores (FIG. 1A). Tremendous progress has been made in recent years in understanding how receptor stimuli regulate different Ca²⁺-channels while less is known about regulatory feedback mechanisms that ensure that the basal Ca²⁺ concentration is kept constant. The tight control of basal Ca²⁺ concentration is relevant for cells as indicated by the numerous diseases that have been associated with prolonged increases or decreases in basal Ca²⁺ concentration. For example, cells derived from Alzheimer disease patients with genetic mutations in preselinin have defects in Ca²⁺ homeostasis (Stutsman, 2005; Zatti et al., 2006) due to changes in the rate of basal Ca²⁺-flux out of the ER Ca²⁺ store (Tu et al., 2006). Changes in basal Ca²⁺ homeostasis have also been associated with diseases such as endothelial dysfunction (Shulman et al., 2005), kidney disease (Thebault et al., 2006), cardiac dysfunction (Ter Keurs and Boyden, 2007), Huntington disease (Bezprozvanny and Hayden, 2004), as well as other neurodegenerative and aging related diseases (Treves et al., 2005; Mattson, 2007; Raza et al., 2007).

Mechanistically, these different disease states are believed to be caused by small but long-term increases or decreases in basal Ca²⁺ concentration that then result in defective Ca²⁺ signaling (Ter Keurs and Boyden, 2007), reduced ER Ca²⁺ concentration and ER stress (Zhang & Kaufman, 2006) or mitochondrial dysfunction (Campanella et al., 2004). Long term changes in basal Ca²⁺ also alter protein degradation (Spira et al., 2001) and transcription (Gallo, 2006) which may indirectly interfere with cell health. The active components that maintain Ca²⁺ gradients in human cells are believed to be four different PM pump isoforms (Pmca's) (Strehler et al., 2007; Guerini et al., 2005) and three PM Ca²⁺ transporters (NaCa-exchangers) (Philipson et al., 2002) as well as three different ER Ca²⁺ pump isoforms (Serca's) (Periasamy, 2007). Less is known about the nature of basal Ca²⁺ fluxes to the cytosol from outside of the cell and from ER Ca²⁺ stores but some Ca²⁺ channels and other Ca²⁺ leak activities have been proposed to possibly play a role (Tu et al., 2006; Pinton and Rizzuto, 2005; Camello et al., 2002). Remarkably, many researchers have observed that basal Ca²⁺ levels in cells change only little in response to large increases or decreases in external Ca²⁺ concentration, arguing that a highly effective feedback control has to exist that stabilizes basal Ca²⁺ concentration (Kusters et al., 2005).

Accordingly, there remains a need in this art for methods to monitor the modulation of basal calcium levels in a cell, screen for agents that modulate basal calcium levels in a cell, and methods for regulating basal calcium levels in a cell. The present invention addresses these and other needs.

### SUMMARY OF THE INVENTION

The invention features methods and compositions for determining the basal calcium level state in a eukaryotic cell. Also provided are methods for identifying an agent (e.g., a gene product or small molecule compound) that modulates basal intracellular calcium levels (e.g., by modulating STIM-2 activity), as well as kits and systems for practicing the subject methods.

Aspects of the invention include methods of assessing the basal calcium level state in a cell including the steps of: providing a cell comprising a STIM-2 polypeptide; detecting a distribution pattern of the STIM-2 polypeptide in the cell; and assessing the basal calcium level state in the cell based on the distribution pattern.

In certain embodiments, the STIM-2 polypeptide comprises a detectible domain.

In certain embodiments, the detectable domain is a fluorescent polypeptide.

In certain embodiments, a punctate STIM-2 distribution pattern is indicative of depleted basal intracellular calcium levels.

In certain embodiments, a diffuse STIM-2 distribution pattern is indicative of levels of basal intracellular calcium that are not depleted.

Aspects of the invention include methods of identifying candidate agents that modulate basal calcium levels in a cell including the steps of: contacting said cell with a candidate agent; assessing basal calcium level changes in said cell as a result of said contacting; and identifying said candidate agent as a modulator of basal calcium levels in said cell based on said assessing.

In certain embodiments, the cells are cultured in calcium sensitizing conditions prior to the contacting.

In certain embodiments, the calcium sensitizing conditions include culturing the cell in high levels of extracellular calcium, and where the agent is identified as modulator of basal calcium levels when the assessed basal calcium level increases after the contacting step.

In certain embodiments, the sensitizing condition include culturing the cell in low levels of extracellular calcium, and where the agent is identified as modulator of basal calcium levels when the assessed basal calcium level decreases after the contacting step.

In certain embodiments, the culturing step comprises culturing the cell in a first and a second distinct sensitizing condition, where the first sensitizing condition comprises culturing the cell in high levels of extracellular calcium and the second sensitizing condition comprises culturing the cell under low levels of extracellular calcium.

In certain embodiments, the cells express a detectible STIM protein and the assessing includes detecting the distribution of the detectible STIM protein.

In certain embodiments, the candidate agent is a nucleic acid.

In certain embodiments, the nucleic acid is a RNAi agent.

In certain embodiments, the candidate agent is a small molecule.

Aspects of the invention include methods of modulating basal calcium levels in a cell including the steps of: contacting the cell with an agent that modulates STIM-2 activity in the cell, where the basal calcium levels in the cell are modulated.

In certain embodiments, modulating STIM-2 activity includes one or more of: modulating STIM-2 calcium binding, modulating STIM-2 aggregation, modulating STIM-2 expression level, and modulating STIM-2-mediated calcium transport.

In certain embodiments, the agent increases STIM-2 activity, thereby increasing basal calcium levels in the cell.

In certain embodiments, the agent decreases STIM-2 activity, thereby decreasing basal calcium levels in the cell.

Aspects of the invention include methods of treating a subject having a condition associated with dysregulated cellular basal calcium levels including administering to the subject an effective amount of an agent that modulates cellular STIM-2 activity, where the condition associated with dysregulated cellular basal calcium levels is treated in the subject.

In certain embodiments, modulating STIM-2 activity includes one or more of: modulating STIM-2 calcium binding, modulating STIM-2 aggregation, modulating STIM-2 expression level, and modulating STIM-2-mediated calcium transport.

In certain embodiments, the condition is characterized by low basal calcium levels and the agent increases the cellular STIM-2 activity.

In certain embodiments, the condition is characterized by high basal calcium levels and the agent decreases the cellular STIM-2 activity.

These and other advantages, aspects, features, and embodiments will be readily apparent to the ordinarily skilled artisan upon reading the present specification.

### BRIEF DESCRIPTION OF THE DRAWINGS

The patent or application file contains at least one drawing executed in color. Copies of this patent or application publication with color drawing(s) will be provided by the U.S. Patent and Trademark Office upon request and payment of necessary fee.

The disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1. Identification of Stim2 as a regulator of basal Ca⁺². (A) Overview of intracellular Ca²⁺ homeostasis. Basal cytosolic Ca²⁺ concentration is controlled by PM as well as ER Ca²⁺ channels and pumps. PMCA in the PM and SERCA in the ER are key active components that pump Ca²⁺ across concentration gradients. (B) Sensitized siRNA screening assay for basal Ca²⁺ regulation. 2400 diced siRNA constructs were individually transfected into HeLa cells cultured in 384 well plates. High and Low extracellular Ca²⁺ exposure (+10 mM and ˜0.1 mM) was used for sensitization. Single cell Ca²⁺ levels were measured using automated image analysis software. (C) Test experiments using a siRNA set targeting Ca²⁺ pumps, channels, and exchangers (performed in duplicate). Deviations from control Ca²⁺ levels are shown in units of standard deviation. (D) Result from the sensitized siRNA screen of the human signaling proteome highlighting Stim2 and CALM1 as primary hits (performed in triplicate). (E) Schematic representation of modular domains found in Stim2. On the luminal side: EF-hand is a Ca²⁺ binding domain and SAM is a conserved protein interaction domain. On the cytosolic side: CC and PB are a coiled-coil and a polybasic region, respectively.

FIG. 2. Stim2 controls basal cytosolic as well as ER Ca²⁺ concentration. (A) Comparison of basal Ca²⁺ levels after siRNA knockdown of Stim2 compared to Stim1. HeLa, HUVEC, and HEK293T cells were transfected with synthetic siRNA against Stim2 and Stim1 as well as diced G13 as a control. N=10 sites; error bars represent std. error. (B) Basal ER Ca²⁺ levels were measured as the Ca²⁺ pool that can be released by addition of the Ca²⁺-ionophore ionomycin. 1 uM ionomycin+3 mM EGTA were added to HeLa cells and the increase in cytosolic Ca²⁺ was measured (Δpeak). Single cell analysis from 3 wells each. (C) Schematic representation of the effects of PMCA1, SERCA2, and Stim2 knockdowns on basal Ca²⁺ levels in the ER and cytosol. (D) Single cell analysis of basal Ca²⁺ concentration as a function of the expression level of YFP-Stim2 versus YFP-Stim1. Cells were transfected for 9 hours with YFP-Stim1, YFP-Stim2, or YFP (as a control). Ca²⁺ levels and YFP construct expression were measured for each cell. YFP fluorescence was normalized to the background in the YFP channel and binned according to increasing YFP levels. (E) Single cell analysis of Ca²⁺-influx triggered by ER Ca²⁺ store-depletion (Ca²⁺-add back experiments). Cells were depleted of ER Ca²⁺ by additions of 1 uM thapsigargin to block SERCA pumps and 3 mM external EGTA to prevent Ca²⁺ influx. Ca²⁺ was added back (to a free concentration of 0.75 mM) at t=0 to measure Ca²⁺ influx rates. Single cells were analyzed in 3 independent wells for each condition.

FIG. 3. Stim2 translocates to ER-PM junctions following ER Ca²⁺ depletion. (A-C) YFP-Stim2 was expressed (˜24 hour) in HeLa (A), HUVEC, (B), and HEK293T (C) cells and confocal images were taken before and 2 minutes after 1 uM thapsigargin addition. (D) Comparison of the distribution of CFP-Stim1 and YFP-Stim2 constructs shows that both co-localize to the previously characterized ER-PM junction sites. (E) Ca²⁺-binding deficient Stim2 (point mutation EF-hand mutant) is already prelocalized to ER-PM junction sites and does not alter its localization after Ca²⁺ store depletion (HeLa cells). (F) Knockdown of the PM Ca²⁺ channel Orai1 significantly reduces the increase in basal Ca²⁺ resulting from Stim2 expression. HeLa cells were transfected for two days with ORAI1 siRNA and transfected for 24 hours with YFP-Stim2. Cells used in the analysis expressed YFP at 7.5 to 15 fold above background. N=10 sites.

FIG. 4. Stim2 translocation and Stim2-mediated Ca²⁺ influx regulated by near basal ER Ca²⁺ concentrations. (A) Stim2 translocates to ER-PM junctions at higher ER Ca²⁺ concentrations compared to Stim1. YFP-Stim2 and CFP-Stim1 were co-tranfected (˜24 hour) and imaged in the same cells. ER Ca²⁺ stores were depleted slowly by extracellular addition of 3 mM EGTA which leads to a loss of ER Ca²⁺ over a period of 30-60 minutes. YFP-Stim2 and CFP-Stim1 distributions are compared before, 2 minutes after and 35 minutes after 3 mM EGTA addition. (B) Analysis of the kinetics of Stim1 and Stim2 translocation to ER-PM junctions upon EGTA addition. 3 mM EGTA was added to HeLa cells and imaged for 60 minutes. Cells were analyzed for puncta content as described in the Materials and Methods section. Average puncta intensity from N=5 cells. (C) Calibration and quantitative model derived from the data in (B). ER Ca²⁺ concentration was calibrated as a function of time after EGTA addition (FIG. 8). The concentration dependence of translocation was then fit to a cooperative oligomerization and translocation model. The scheme shows the key features of our model that includes, in addition to the differential Ca²⁺ sensitivity, an oligomerization and translocation process with a cooperativity of 5-8 for Stim2 and Stim1 activation. (D-E) Functional comparison supporting that Stim2 activity is suppressed by higher ER-Ca²⁺ levels compared to Stim1. Basal Ca²⁺ levels were measured as a function of the expression level of different Stim1 and Stim2 constructs (12 h of transfection). Normal (dashed lines) and reduced (solid lines) ER Ca²⁺ levels were used to probe for the respective Stim1 and Stim2 Ca²⁺ sensitivities. Expression of EF-hand mutant Stim2 and Stim1 constructs (black) were employed as a reference of Ca²⁺-insensitive and constitutively active proteins. Wildtype Stim1 (green) or Stim2 (red) expression showed for normal ER Ca²⁺ a marked difference in the profile compared to the one of the EF-hand mutants. In contrast, Stim2 but not Stim1 matched the profile of the EF-hand mutant at reduced ER Ca²⁺ levels, suggesting that reduced ER Ca²⁺ levels can still suppress Stim1 but not Stim2 activity. N=20 wells.

FIG. 5. Control experiment showing that Stim2 activates Ca²⁺-influx in cells treated with Stim1 siRNA. (A) The Stim2-triggered increase B-SOC and basal Ca²⁺ is not affected by knockdown of Stim1. Stim1 knockdown is compared to a GL3 control siRNA. N=10 sites. (B-C) Experiments showing that Stim2-triggered R-SOC is not affected by Stim1 knockdown and Stim1-triggered R-SOC is not significantly affected by Stim2 knockdown. Ca²⁺ addback experiments were performed in cells expressing increasing levels of Stim2 (B) and Stim1 (C). All single cells were analyzed in two wells for each condition. (D) Schematic representation of the regulation of B-SOC and R-SOC by Stim2 and Stim1 under basal and receptor-stimulated conditions, respectively.

FIG. 6. Model for Stim2 function in basal Ca²⁺ homoestasis. Schematic representations of the stabilization of basal cytosolic and ER Ca²⁺ concentrations by negative feedback. Stim2 and Orai1 are at the center of a negative Ca²⁺ feedback circuit that connects changes in basal ER and cytosolic Ca²⁺ concentration to Ca²⁺ influx.

FIG. 7. Timing of Stim1 and Stim2 puncta formation upon thapsigargin addition. 1 μM thapsigargin was added to HeLa cells and imaged for 220 seconds. Images were then analyzed for puncta content as in described in Materials and Methods section. N=4 cells each.

FIG. 8. Calibration of the ER Ca²⁺ content at different time-points following external addition of EGTA. 3 mM EGTA was added to wells at time=0 min. Ionomycin was added to different wells at the indicated time points. The measured ΔCa²⁺ peak heights were fit to an exponential decay.

### DEFINITIONS

Unless defined otherwise, all technical and scientific terms used herein have the same meaning as commonly understood by those of ordinary skill in the art to which this invention pertains. Any methods and materials similar or equivalent to those described herein can be used in the practice or testing of the present invention, the described methods and materials being exemplary.

A “distribution pattern” refers to a pattern of expression of a moiety (e.g., a STIM-2 protein) in a cell, for example on an endoplasmic reticulum membrane. A distribution pattern is determined by observing a detectable signal associated with moiety of interest (e.g., a STIM-2 protein). For example, a punctate distribution pattern of STIM-2 in a cell is indicative of depleted basal intracellular calcium levels in a cell. Conversely, a diffuse distribution pattern of STIM-2 is indicative of levels of basal intracellular calcium that are not depleted.

The term “STIM-2 activity” refers generally to the amount of STIM-2-mediated calcium transport that is occurring in a cell, where an increase in STIM-2 activity leads to an increase in STIM-2-mediated calcium transport in a cell (e.g, where STIM-2 is bound to calcium and in a punctate distribution pattern) and where a decrease in STIM-2 activity leads to a decrease in STIM-2-mediated calcium transport in a cell (e.g, where STIM-2 is bound to calcium and in a punctate distribution pattern). STIM-2 activity in a cell can be increased or decreased (or modulated) in a number of ways, including (but not limited to) increasing or decreasing the amount of STIM-2 in a cell by contacting a cell with an agent that modulates STIM-2 calcium binding, aggregation, expression level, calcium transport, etc. As described herein below, a decrease in STIM-2 activity can lead to a reduction in basal calcium levels in a cell whereas an increase in STIM-2 activity can lead to an increase in basal calcium levels in a cell.

As used herein “STIM-2 state” refers to the distribution of a STIM-2 polypeptide within a cell (e.g., an aggregate of STIM-2 polypeptides giving a punctate pattern, or as discrete, non-aggregated STIM-2 polypeptides giving a diffuse pattern), which state depends on the calcium binding state of the STIM-2 polypeptide (e.g., presence or absence of bound calcium).

Unless indicated otherwise either specifically or by context, “intracellular calcium” generally refers to “cytosolic calcium” in a cell; “intracellular store calcium”, “store calcium”, “stored intracellular calcium”, or “intracellular calcium stores”, and the like, refers to calcium in sequestered in the ER or other organelles in a cell.

“Low level of intracellular store calcium” in a cell as used herein refers to a calcium state in the store that is depleted relative to normal, and generally refers to a state in the cell in which a STIM polypeptide would form puncta in a cell membrane, e.g., in the endoplasmic reticulum membrane. In general “normal intracellular store calcium” refers to a state of the cell in which the concentration of calcium in intracellular stores is about 50 μM to about 400 μM. In general, under physiological conditions, normal cytosolic calcium concentrations are about 100 nM, and normal extracellular calcium concentrations are generally about 1 mM.

The term “modulates”, as in, for example, “modulates basal calcium levels”, “modulates STIM-2 activity”, or “modulates STIM-2 distribution pattern”, particularly in reference to an agent (e.g., a candidate agent) is meant that the agent directly or indirectly effects an increase or decrease in the associated cellular event.

The term “stimulus” refers to an environmental condition, e.g., exposure to an agent, temperature, light, osmolarity, and the like, which may elicit a response, e.g., modulation of calcium signaling which can be associated with a change in intracellular store calcium levels, SOC influx, basal calcium levels and the like.

The term “agent” includes any substance, molecule, element, compound, entity, or a combination thereof. It includes, but is not limited to, e.g., proteins (including antibodies), oligopeptides, small organic molecules, polysaccharides, polynucleotides (e.g., DNA or RNA, including polynucleotides encoding a gene product of interest, or which act as a cell modulator without transcription or without translation), and the like. It can be a natural product, a synthetic compound, or a chemical compound, or a combination of two or more substances. Unless otherwise specified, the terms “agent,” “substance,” and “compound” can be used interchangeably.

The term “analog” is used herein to refer to a molecule that structurally resembles a molecule of interest but which has been modified in a targeted and controlled manner, by replacing a specific substituent of the reference molecule with an alternate substituent. Compared to the starting molecule, an analog may exhibit the same, similar, or improved utility. Synthesis and screening of analogs, to identify variants of known compounds having improved traits (such as higher potency at a specific receptor type, or higher selectivity at a targeted receptor type and lower activity levels at other receptor types) is an approach that is well known in pharmaceutical chemistry.

The term “biological preparation” refers to biological samples taken in vivo or in vitro (either with or without subsequent manipulation), as well as those prepared synthetically. Representative examples of biological preparations include cells, tissues, solutions and bodily fluids, lysates of natural or recombinant cells, and samples derived from such sources.

As used herein, the term “functional derivative” of a native protein or a polypeptide is used to define biologically active amino acid sequence variants that possess the biological activities (either functional or structural) that are substantially similar to those of the reference protein or polypeptide.

The terms “substantially pure” or “isolated,” when referring to proteins and polypeptides denote those polypeptides that are separated from proteins or other contaminants with which they are naturally associated. A protein or polypeptide is considered substantially pure when that protein makes up greater than about 50% of the total protein content of the composition containing that protein, and typically, greater than about 60% of the total protein content. More typically, a substantially pure or isolated protein or polypeptide will make up at least 75%, more preferably, at least 90%, of the total protein. Preferably, the protein will make up greater than about 90%, and more preferably, greater than about 95% of the total protein in the composition.

The terms “nucleic acid molecule” and “polynucleotide” are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers.

“Encoded by” refers to a nucleic acid sequence which codes for a polypeptide sequence, wherein the polypeptide sequence or a portion thereof contains an amino acid sequence of at least 3 to 5 amino acids, more preferably at least 8 to 10 amino acids, and even more preferably at least 15 to 20 amino acids from a polypeptide encoded by the nucleic acid sequence. Also encompassed are polypeptide sequences that are immunologically identifiable with a polypeptide encoded by the sequence.

“Sequence identity” refers to a number of residues shared between a query amino acid sequence and a reference amino acid sequence (or between a query nucleotide sequence and a reference nucleotide sequence) over a region of alignment. In general, sequence identity is calculated based on a reference sequence, which may be a subset of a larger sequence, such as a conserved motif, coding region, flanking region, etc. A reference sequence will usually be at least about 6 amino acids long, usually at least about 10 amino acids long (or about 18 nt long, more usually at least about 30 nt long), and may extend to the complete sequence that is being compared. In general, percent sequence identity is calculated by counting the number of residue matches (e.g., nucleotide residue or amino acid residue) between the query and test sequence and dividing total number of matches by the number of residues of the individual sequences found in the region of strongest alignment. Thus, where 98 residues of a 100 residue query sequence matches a test sequence, the percent identity would be 98 divided by 100, or 98%. Algorithms for computer-based amino acid and nucleotide sequence analysis are known in the art, such as BLAST (see, e.g., Altschul et al., J. Mol. Biol., 215:403-10 (1990)), particularly the Smith-Waterman homology search algorithm as implemented in MPSRCH program (Oxford Molecular). For the purposes of this invention, a preferred method of calculating percent identity is the Smith-Waterman algorithm, using the following: Global DNA sequence identity must be greater than 65% as determined by the Smith-Waterman homology search algorithm as implemented in MPSRCH program (Oxford Molecular) using an affine gap search with the following search parameters: gap open penalty, 12; and gap extension penalty, 1.

A “vector” is capable of transferring gene sequences to target cells. Typically, “vector construct,” “expression vector,” and “gene transfer vector,” mean any nucleic acid construct capable of directing the expression of a gene of interest and which can transfer gene sequences to target cells. Thus, the term includes cloning, and expression vehicles, as well as integrating vectors.

As used herein, “recombinant” has the usual meaning in the art, and refers to a polynucleotide synthesized or otherwise manipulated in vitro (e.g., “recombinant polynucleotide”), to methods of using recombinant polynucleotides to produce gene products in cells or other biological systems, or to a polypeptide (“recombinant protein”) encoded by a recombinant polynucleotide.

The term “recombinant” when used with reference to a cell indicates that the cell replicates a heterologous nucleic acid, or expresses a peptide or protein encoded by such a heterologous nucleic acid. Recombinant cells can contain genes that are not found within the native (non-recombinant) form of the cell. Recombinant cells can also contain genes found in the native form of the cell wherein the genes are modified and re-introduced into the cell by artificial means. The term also encompasses cells that contain a nucleic acid endogenous to the cell that has been modified without removing the nucleic acid from the cell; such modifications include those obtained by gene replacement, site-specific mutation, and related techniques.

A “heterologous sequence”, “heterologous nucleic acid”, “heterologous polypeptide” or “heterologous amino acid sequence” as used herein, is one that originates from a source foreign to the particular host cell, or, if from the same source, is modified from its original form. Thus, a heterologous nucleic acid in a host cell includes nucleic acid that, although being endogenous to the particular host cell, has been modified (e.g., so that it encodes an amino acid sequence different from that of the endogenous nucleic acid, to a nucleic acid to provide a sequence not normally found in the host cell, and the like). Modification of the heterologous sequence can occur, e.g., by treating the DNA with a restriction enzyme to generate a DNA fragment that is capable of being operably linked to the promoter or by operably linking the DNA to a heterologous promoter to provide an expression cassette that is not endogenous to the host cell. Techniques such as site-directed mutagenesis are also useful for modifying a heterologous nucleic acid.

The term “operably linked” refers to functional linkage between nucleic acids to provide a desired activity, e.g., a functional linkage between a nucleic acid expression control sequence (such as a promoter, signal sequence, or array of transcription factor binding sites) and a second polynucleotide, wherein the expression control sequence affects transcription and/or translation of the second polynucleotide. “Operably linked” in the context of a polypeptide refers to a functional linkage between amino acid sequences (e.g., of different domains) to provide for a described activity of the polypeptide (e.g., a nuclear localization signal is operably linked to a heterologous amino acid sequence to provide to association of the fusion protein with the nucleus in a mammalian cell).

A “recombinant expression cassette” or simply an “expression cassette” is a nucleic acid construct, generated recombinantly or synthetically, that has control elements that are capable of affecting expression of a structural gene that is operably linked to the control elements in hosts compatible with such sequences. Expression cassettes include at least promoters and optionally, transcription termination signals. Typically, the recombinant expression cassette includes at least a nucleic acid to be transcribed and a promoter. Additional factors necessary or helpful in effecting expression can also be used as described herein. For example, transcription termination signals, enhancers, and other nucleic acid sequences that influence gene expression, can also be included in an expression cassette.

As used herein, “contacting” has its normal meaning and refers to combining two or more entities (e.g., two proteins, a polynucleotide and a cell, a cell and a candidate agent, etc.). Contacting can occur in vitro (e.g., two or more agents [e.g., a test compound and a cell lysate] are combined in a test tube or other container) or in situ (e.g., two polypeptides can be contacted in a cell by coexpression in the cell, of recombinant polynucleotides encoding the two polypeptides), in a cell lysate.

By “genetic transformation” is meant a permanent or transient genetic change induced in a cell following incorporation of exogenous nucleic acid (e.g., DNA or RNA exogenous to the cell). Genetic change can be accomplished by, for example, incorporation of exogenous DNA into the genome of a host cell, by transient or stable maintenance of the exogenous DNA as an episomal element, or by transient introduction of an exogenous RNA into the host cell. Where the cell is a mammalian cell, a permanent genetic change is generally achieved by introduction of the DNA into the genome of the cell.

A “biological sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents; washed; or enrichment for certain cell populations, such as CD4+ cells, T lymphocytes, macrophages, peripheral blood mononuclear cells (PBMC), and the like. The term “biological sample” encompasses a clinical sample, and also includes cells in culture, cell supernatants, tissue samples, organs, bone marrow, and the like.

### DETAILED DESCRIPTION OF THE INVENTION

Provided herein are methods and compositions related to the identification of STIM-2 as a regulator of basal calcium levels in eukaryotic cells. Methods and compositions are provided for identifying agents that modulate basal calcium levels in a cell, assessing basal calcium levels in a cell, and modulating basal calcium levels in a cell, e.g., for treating pathological conditions associated with dysregulated basal calcium levels. The invention also provides kits and systems for practicing the subject methods.

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the biosensor” includes reference to one or more biosensor and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Various biochemical and molecular biology methods referred to herein are well known in the art, and are described in, for example, Sambrook et al., Molecular Cloning: A Laboratory Manual, Cold Spring Harbor Press, N.Y. Second (1989) and Third (2000) Editions, and Current Protocols in Molecular Biology, (Ausubel, F. M. et al., eds.) John Wiley & Sons, Inc., New York (1987-1999).

As summarized above, methods and compositions are provided herein that are related to the inventor's findings that STIM-2, unlike STIM-1, is a regulator of basal calcium levels in eukaryotic cells. Coupled with an understanding of the mechanism of action of STIM polypeptides, including both STIM1 and STIM2 (e.g., as described in co-pending U.S. patent application Ser. No. 11/446,010, filed Jun. 2, 2006, and published Dec. 21, 2006 as U.S. Application Publication Number US2006/0286605, incorporated herein in its entirety by reference), we provide methods and compositions for identifying agents that modulate basal calcium levels in a cell, assessing basal calcium levels in a cell, and modulating basal calcium levels in a cell, e.g., for treating pathological conditions associated with dysregulated basal calcium levels. The invention also provides kits and systems for practicing the subject methods.

As provided in greater detail below, the subject methods of the present invention are practiced using a cell containing a STIM-2 protein (e.g., either endogenous or exogenous) that provides a detectable pattern (e.g., a fluorescent pattern) indicative of basal calcium levels within the cell. Assessing the detectable pattern in real time or at a given time point can be used to determine the basal calcium state. Accordingly the subject methods can be used for identifying an agent (e.g., a gene product or small molecule compound) that modulates the basal calcium level in a cell. Such identified agents may serve as therapeutic targets and/or pharmaceutical compositions suitable for treating pathological conditions associated with dysregulated basal calcium levels, and thus modulates calcium signaling, calcium store levels, and/or the cell's state of calcium influx.

For example, in one embodiment the STIM-2 protein is an engineered protein that contains a fluorescent polypeptide operably linked to the calcium responsive polypeptide STIM-2. When the EF hand domain of this STIM-2 protein is bound by calcium, it remains dispersed throughout a cellular membrane, particularly the ER membrane. In this state, the protein provides as a granulated, dispersed pattern, generally referred to herein as a “diffuse” pattern. When the STIM-2 senses low calcium at its EF hand domain (e.g., due to decreased binding of the EF hand domain to calcium), STIM-2 aggregates in membranes, providing a punctate pattern.

The methods of the subject invention are simple and rapid to perform. The methods provide a highly sensitive and specific assay, allowing determination of the calcium biosensor state and thus a cell's state of intracellular store calcium, SOC influx state, and the like. This assay not only offers a powerful tool to interpret calcium biosensor information, but can also be used to identify new modulators of calcium signaling, calcium binding to calcium biosensor polypeptides, mobilization of calcium from intracellular stores, mobilization of calcium to replenish stores (e.g., as a result of calcium mobilization into stores following SOC influx), where such modulators can range from small molecules, endogenous or nonendogenous gene products, and environmental stimuli.

The following description provides guidance for making and using the compositions of the invention, and for carrying out the methods of the invention.

Screen for Identifying Agents that Modulate Basal Calcium Levels in a Cell

The present invention provides a screen for agents that regulate basal calcium levels in a eukaryotic cell.

In general, the basal calcium screen includes contacting the cells of interest to a candidate agent for a period of time sufficient for the agent to have an effect in the cell followed by measuring the basal calcium levels in the cells and assessing the basal calcium level changes in the cell after the contacting step. The time sufficient for the agent to have an effect will depend on the agent(s) being screened, where the contacting step may take from hours to days (e.g., approximately 1 hour for small molecule candidate agents, 2 days for siRNA candidate agents). Measuring the calcium levels can be achieved using any convenient method, including loading the cells with a calcium dye (e.g. Fura-2) or employ cells expressing a fluorescent protein calcium indicator (e.g. cameleon or YFP-STIM, which is discussed in further detail below).

In certain embodiments, the measuring and/or assessing step includes analyzing data using automated imaging software. In certain of these embodiments, the sensitivity of the measuring and assessing steps is in the nanomolar range (meaning that nanomolar changes in calcium levels in the cell can be detected), and as such, normalization and correction for background illumination and/or plate-effects is carefully controlled for. For example, it has been found that in some experiments, the position of the cell on the plate can affect the measured calcium levels in the cell (e.g., cells on one side of a plate may appear to have higher basal calcium levels compared to cells on the opposite side). In addition, one plate of cells in an assay (e.g., a high throughput assay of the present invention) may appear to have a lower average calcium level than another. Therefore, in certain embodiments, background/basal calcium levels in cells being assayed should be controlled within a single well (or plate) and/or between distinct wells/plates.

Once calcium levels in the cells are measured and assessed, agents that regulate basal levels of calcium are identified. In general, an agent is identified as a regulator of basal calcium levels in a cell if it significantly reduces or increases basal calcium levels in a cell.

In certain embodiments, the screen includes culturing a cell (or cell population) under one or more calcium sensitizing conditions (with each sensitizing condition representing an independent culture), contacting the calcium sensitized cells to a candidate agent, and assessing basal calcium level changes in the cell after the contacting step. The sensitization culture serves to push the Ca²⁺ homeostatic control system towards its limits so that the effect of an agent on basal calcium levels is more readily observed. The cells may be any cell for which basal calcium regulating agents are sought, including virtually any eukaryotic cell (e.g., mammalian cells, insect cells, worm cells, yeast cells, etc.), including primary cells, transformed cells, genetically modified cells, etc. In certain embodiments, the cells are transformed human cells (e.g., HeLa cells).

In certain embodiments, the calcium sensitizing conditions include cultures that have excess extracellular calcium and/or cultures that have low extracellular calcium, where by excess extracellular calcium is meant levels of Ca²⁺ in the culture medium ranging from 5.0 mM to 25.0 mM and by low extracellular calcium is meant levels of Ca²⁺ in the culture medium ranging from 0.02 mM to 0.2 mM. In certain embodiments, the modified screen is set up with multiple cell cultures, each under distinct calcium sensitizing culture conditions (e.g., a first cell culture having high extracellular calcium and a second culture having low extracellular calcium). Such independent cultures can be set up simultaneously or in series, depending on the constraints of the assay system being employed. The cells may be cultured under the calcium sensitizing conditions for any amount of time deemed sufficient to appropriately render them sensitized, including from hours to days. In certain embodiments, a cell is cultured under sensitization culture condition for a time period ranging from 4 hours to 3 days.

As indicated above, agents that can be screened in this assay include any substance, molecule, element, compound, entity, or a combination thereof. In certain embodiments, the agent is an agent that is known or predicted to affect the expression of one or more genes in a cell, including RNAi agents (e.g., an siRNA library), gene expression vectors (e.g., in an expression library), and the like.

After sensitization and agent contact, the change in calcium level in each the cell is determined. Any convenient method for assaying the changes in calcium levels in the cell can be employed, including the use of fluorescent Ca²⁺ binding agents (e.g., Fura-2 and the like). In certain embodiments, the calcium level change is read using an automated fluorescence imaging system.

Once calcium levels in the cells are assessed, agents that regulate basal levels of calcium are identified. In general, an agent is identified as a regulator of basal calcium levels in a cell if it reduces basal calcium levels in a cell cultured under low levels of extracellular calcium and/or if it increases basal calcium levels in a cell cultured under high levels of extracellular calcium.

### Stim-2 is a Regulator of Basal Calcium Levels

As described in detail in the Examples section, the inventors have identified STIM-2 as a regulator of basal calcium levels in cells using a version of the modified screening assay detailed above. Biopolymeric sequences for STIM-2 (stromal interaction molecule 2; also known as FLJ39527, KIAA1482, and stromal interaction molecule 2 precursor) include the following: UniProt ID numbers Q9P246, Q96BF1, and Q9BQH2; NCBI UniGene ID number 57620; NCBI RefSeq ID numbers NP_{—}065911 and NM_{—}020860; and NCBI Accession Numbers CAB66512 and BAB14545. Homologs of STIM-2 include, but are not limited to, the following: Mus musculus (e.g., NCBI UniGene ID number 116873), Pan troglodites (e.g., NCBI UniGene ID number 461155), Gallus gallus (e.g., NCBI UniGene ID number 422799), Rattus norvegicus (e.g., NCBI UniGene ID number 117087), and Canis familiaris (NCBI UniGene ID number 479120).

Without being held to theory, the presence or absence of calcium bound to the EF-hand domain of STIM-2 modulates changes in its localization in the cell. In particular, when basal calcium levels are low, and thus the EF-hand domain is not bound by calcium, STIM-2 forms aggregates in cell membranes, e.g., in the ER plasma membrane, allowing an influx of calcium into the cytoplasmic compartment. When basal calcium levels are relatively higher and calcium is bound to the EF hand domain, the CBP does not aggregate and thus is spread in a more diffuse pattern in cell membranes, e.g., in the ER plasma membrane, and does not allow calcium influx into the cytoplasm. Therefore, detecting the distribution of endogenous STIM-2 (or a modified STIM-2 comprising a detectible domain, as described below) can be used as a surrogate marker for determining basal calcium levels in a cell as well as whether an agent modulates basal calcium levels in cell (e.g., by modulating that activity of STIM-2).

Detectible STIM-2

As indicated above, the detectible STIM-2 polypeptide of the invention can be a naturally-occurring STIM-2 polypeptide, or a STIM polypeptide modified to contain a detectable domain. In one embodiment, the STIM-2 comprises an amino acid sequence of STIM2 polypeptide as a “backbone”, with a heterologous detectable domain provided in the backbone, e.g., by insertion and/or replacement of amino acids in the STIM-2 polypeptide sequence, usually at a position N-terminal to the EF hand domain (e.g., in the precursor, usually at a position between the signal peptide and the EF hand domain). In other embodiments, a region of the STIM polypeptide endogenous to the polypeptide serves as a detectable domain (e.g., a region of the polypeptide that can be specifically bound by an anti-STIM-2 antibody). Of particular interest are STIM-2 polypeptides modified to contain a detectable domain at the N-terminus, preferably N-terminal of the EF hand domain, wherein the detectable domain is a fluorescent polypeptide.

STIM-2 proteins of the invention include those in both a precursor form (or “immature” form) as well as in the mature or “processed” form. Detectible STIM-2 polypeptides of the present invention include a signal peptide to facilitate delivery of the polypeptide to the appropriate membrane. This signal peptide is cleaved during processing, and thus is absent in the mature form. Thus, in general, constructs suitable for expression of STIM-2 in a cell according to the invention include a signal peptide. Because the invention contemplates both constructs suitable for production of detectible STIM-2 in a host cell, as well as host cells that express the immature and mature forms of detectible STIM-2 (e.g., modified to include a detectible domain, e.g., a fluorescent peptide), the invention includes precursor and processed STIM-2 which contain and lack the signal peptide, respectively.

Generally, where the STIM-2 is structured to include a heterologous detectable domain, the detectable domain is positioned at the N-terminus of the polypeptide, and usually with the C-terminus of the detectable domain being positioned about 40 to 50 amino acids, usually about 45 amino acids, N-terminal of the EF hand domain. In the precursor form, the heterologous detectable domain is preferably positioned between the signal peptide and the EF hand domain. In general, in the precursor form of STIM-2, the signal peptide is provided N-terminal of the detectable domain, which signal peptide is usually from about 20 to 25 amino acids, usually about 22 amino acids. In exemplary embodiments, a region of usually about 30, 35, or 40 amino acids separates the C-terminus of the EF hand domain from the N-terminus of the SAM domain. The C-terminus of the SAM domain is separated from the N-terminus of the transmembrane domain by about 10 to 20 usually about 15 amino acids. The C-terminus of the transmembrane domain is separated from the N-terminus of the ERM domain by about 15 to 25 amino acids, usually by about 20 amino acids.

The detectable domain can be flanked by one or more linkers, which can be for example, from about 5 to 15, from 10 to 15, usually from about 6 to 12 amino acids in length, and can be about 20 amino acids or more, and, where flanking the detectable domain, are selected independently as to length and sequence. Linkers should be selected so that they do not substantially affect function of STIM-2, e.g., linkers should lack a functional domain (e.g., a region relatively rich for positively or negatively charged amino acids) that may affect trafficking of the protein.

Any detectable domain known in the art is suitable for use in the calcium biosensor polypeptides of the present invention. A suitable detectable domain will generally be one that can be expressed in a desired host cell and will readily provide a detectable signal that can be assessed qualitatively and/or quantitatively, and can be detected directly or indirectly. Exemplary detectable domains include fluorescent polypeptides, wherein the fluorescent polypeptides include, but are not limited to, yellow fluorescent protein (YFP), cyan fluorescent protein (CFP), GFP, mRFP, RFP (tdimer2), HCRED, and the like, or variants thereof (e.g., fluorescent proteins modified to provide for enhanced fluorescence or a shifted emission spectrum, e.g., enhanced YFP). Further suitable fluorescent polypeptides, as well as specific examples of those listed herein, are provided in the art and are well known. “Fluorescent polypeptide” or “fluorescent polypeptide domain” as used herein is thus meant to encompass wild-type and modified fluorescent polypeptides. Exemplary non-fluorescent detectable domains include immunodetectable epitopes, such as FLAG, His tags, and the like. It should be noted that where the detectable domain is an immunodetectable domain, detection generally involves permeabilizing the cells (e.g., fixing the cells or treating the cells with a detergent) and contacting the cells with a detectably labeled antibody that specifically binds the immunodetectable domain. Alternatively, binding of the anti-immunodetectable domain antibody can be accomplished using a secondary antibody that is detectably labeled.

In some embodiments, more that one detectible STIM protein is present in a single cell (e.g., two detectible STIM-2 proteins or a detectible STIM-2 protein and a detectible STIM-1 protein). In these embodiments, the detectable domain of each STIM protein may be selected so that each has a detectably different signal. For example, in such embodiments comprising fluorescent polypeptide labels, a first STIM-2 (e.g., one having wild type activity) and a second STIM-2 (e.g., one having non-wild type activity, e.g., a non-functional mutant) are designed to have detectably distinct emission spectra to facilitate detection of a distinct signal from each biosensor (e.g., through use of different filters in the imaging system). As another example, the first STIM protein may be a distinctly labeled STIM-2 protein and the second STIM protein may be a distinctly labeled STIM-1 protein. In such embodiments, the relative distribution pattern of the STIM-2 and STIM-1 proteins may be used to differentiate basal calcium level changes in a cell from inducible calcium level changes in a cell (e.g., in assays for agents that modulate calcium levels in cells).

Guidance as to amino acid variations (e.g., amino acid substitutions, deletions, insertions, and the like) in STIM-2 proteins of the invention are described in detail in co-pending U.S. patent application Ser. No. 11/446,010, filed Jun. 2, 2006, and published Dec. 21, 2006 as U.S. Application Publication Number US2006/0286605, incorporated herein in its entirety (see, e.g., FIG. 10 and its description in the text). In brief, guidance can be provided by alignment of the human amino acid sequences of STIM-1 and STIM-2 with one another as well as by alignment of amino acid sequences of STIM-1 and/or STIM-2 polypeptides with the amino acid sequences of STIM polypeptides that are allelic variants from the same organism or are from other sources. Based on such alignments, a detectible STIM-2 polypeptide may be a modified STIM-2 protein that contains a detectable domain positioned at the N-terminus, preferably N-terminal to the EF hand domain and, in the precursor form, more preferably between the signal peptide and the EF hand domain. Further, such alignments provide guidance to the ordinarily skilled artisan regarding areas of the amino acid sequences within different portions of the STIM polypeptides that can tolerate amino acid changes (e.g., insertions, deletions, substitutions (e.g., conservative or non-conservative amino acid changes)) without loss of relevant function for production of detectible STIM proteins of the invention (as well as production of mutant (e.g., non-functional) detectible STIM proteins of the invention).

Sequences of a large number of STIM polypeptides have been described For example, the amino acid sequences of STIM polypeptides of human (e.g., STIM1: GenBank Acc Nos. NP_{—}003147.2, HSU52426, NM_{—}003156.2, BC021300.2, AY399210.1; Q13586; STIM2: NP_{—}065911, Q9P246, AAH15659, AAH57231, AAK82337), non-human primate (e.g., STIM1: GenBank Acc. No. AY399211), bovine (STIM1: GenBank Acc. No. BT021898, AAX46745), mouse (e.g., STIM1: GenBank Acc. Nos. MMU47323, AK041944.1, BC021644.1, NM_{—}009287.2, AY399212.1 and P70302, NP_{—}033313, AAH21644; STIM2: XP_{—}132038, P83093, AAH43455, AAK823390), rat (e.g., STIM1: GenBank Acc. No. XM_{—}341896), drosophila (e.g., STIM: NP_{—}996470.1), and C. elegans (e.g., STIM: NP_{—}741073.1, NP_{—}741074.1) origin are known in the art, as are the amino acid sequence of other STIM polypeptides.

Additional guidance as to amino acid variations in the detectible STIM-2 proteins of the invention is provided from the knowledge of the functional domains present in the STIM-2 polypeptides that serve as the detectible STIM-2 protein backbones (see, e.g., Williams et al., Biochim Biophys Acta. 2002 Apr. 1; 1596(1):131-7; and Wiliamsn et al., Biochem J. 2001 Aug. 1; 357(Pt 3):673-85. For example, EF hand domains are well-characterized and well known in the art. Generally, one or more aspartic acid residues present in the EF hand domain are important for calcium binding, as illustrated in the Examples below with respect to the CIM polypeptide of the invention. The relationship between structure and function in EF hand domains has been studied extensively, and modifications in the amino acid sequences that can be made without affecting calcium binding affinity are well known. See, e.g., Gulati, et al. FEBS Lett. 1989 May 8; 248(1-2):5-8; Kawasaki et al. Biometals. 1998 December; 11(4):277-95; InterPro Accession Number IPR002048. SAM domains (sterile alpha motif domains) are also well characterized in the art (see, e.g., InterPro Accession Number IPR001660; see also Wiliams et al. Biochim Biophys Acta. 2002 Apr. 1; 1596(1):131-7, discussing analysis of SAM domain in STIM1). ERM domains (E for ezrin, R for radixin and M for moesin) are also well characterized as widespread protein modules that can be involved in localizing proteins to the plasma membrane and crosslinking actin filaments with plasma membranes (InterPro Accession Number IPR000798).

Thus with reference to a detectible STIM-2 polypeptide having a human STIM-2 polypeptide as a backbone, the detectible STIM-2 proteins of the invention can share, for example, 75%, 80%, 85%, 90%, 95% or greater amino acid sequence identity across the portion of the detectible STIM-2 derived from a human STIM-2 polypeptide.

### Detectible STIM-2 Mutants

As indicated above, detectible STIM-2 proteins include mutants of STIM-2 that lack certain functional characteristics. For example, detectible STIM-2 mutants of the present invention can be fusion proteins comprising: 1) a signal peptide (in the precursor form), 2) a detectable domain, usually a heterologous detectable domain, such as a fluorescent polypeptide domain; and 3) a mutant EF-hand domain, which is modified to have a decreased binding affinity for calcium, 4) a SAM domain, 5) a transmembrane domain, and 6) an ERM domain. The modified EF-hand domain causes the detectible STIM-2 mutant to aggregate, thus forming puncta and the punctate pattern, regardless of intracellular stored calcium levels. Accordingly, such detectible STIM-2 mutants will provide a detectable punctate pattern which is not affected by, and thus does not change, with changes in basal calcium levels.

Except for the EF hand domain, the other domains of the detectible STIM-2 protein are similar or the same as those for CBPs as described above. The EF hand domain is well characterized, and modifications to provide for decreased binding of calcium relative to an unmodified EF hand domain are known in the art. For example, substitution or deletion of an aspartic acid residue in the EF hand domain, as described in the examples below, can provide for the desired decreased calcium binding affinity.

The detectibel STIM-2 mutant and the detectible STIM-2 protein (with wild-type activity and distribution pattern) can be provided in the same or different host cells. Where these proteins are provided in the same host cell, e.g., so that the detectible STIM-2 mutant can serve as an internal control for a punctate pattern of expression, the detectable domains of the wild-type and mutant STIM-2 proteins are selected so that they can be distinguished based on the respective detectable signals. For example, in such embodiments comprising fluorescent polypeptide labels, the fluorescent domain of the detectible STIM-2 mutant is selected to have a different emission spectra compared to that of the fluorescent domain of the detectible STIM-2 protein to facilitate detection of a distinct signal from each (e.g., through use of different filters in the imaging system).

Where the detectable domains are fluorescent, the fluorescent polypeptides can be selected so that two different emission spectra are detected in different patterns (e.g., a diffuse pattern for the detectible STIM-2 protein, and a punctate pattern for the detectible STIM-2 mutant when the cell is in a normal basal calcium state). When the basal state changes, e.g., the cell is in a low basal calcium state, the first and second emission spectra are colocalized due to aggregation of the detectible STIM-2 protein. In this way, a decrease in basal calcium in the cell can be determined by assessing colocalization of two different emission spectra. In general, images of the “wild-type” and mutant STIM-2 protein patterns are taken using two different channels using a fluorescence microscope, and images processed to overlay the two images (e.g., using a colocalization function commonly found in image processing software). Methods for assessing colocalization of the two images can be accomplished using methods and tools readily available in the art, e.g., MetaMorph™ (Universal Imaging). Colocalization of the “wild-type” and mutant STIM-2 proteins images can be assessed qualitatively or quantitatively.

Accordingly, in one embodiment, the invention provides an array that includes at least one host cell containing a detectible STIM-2 mutant-encoding construct of the invention on at least one defined spot on the array (e.g., within at least one well of a multi-well array). The detectible STIM-2 mutant-containing host cell can serve as a reference cell for a punctate pattern associated with a low basal calcium levels in the cell. In another embodiment, the invention provides an array that includes at least one host cell containing a detectible STIM-2 protein-encoding construct (a “wild-type” STIM-2) of the invention on at least one defined spot on the array (e.g., within at least one well of a multi-well array). In still another embodiment, the invention provides an array that includes at least one host cell containing a detectible STIM-2 mutant-encoding construct and a detectible STIM-2 protein-encoding construct, where each STIM-2 has different detectable domain which provide for distinct signals. In this latter embodiment, the detectible STIM-2 mutant serves as an internal control for the punctate pattern associated with a low basal calcium levels in the cell.

The methods of the invention are amenable to monitoring cells in culture using any suitable microscopic method. In one embodiment, the detectable domain of the STIM-2 protein is fluorescent. Where the detectable domains are immunodetectable, detection can be accomplished using a labeled primary antibody that specifically binds the detectable domain of the STIM-2 protein. Alternatively, the primary antibody can be unlabeled, and binding of primary antibody detected using a secondary labeled antibody. Variations on antibody-based detection systems are known in the art, and can be readily adapted to the invention, as will be apparent to the ordinarily skilled artisan.

Where the detectable domain of the detectible STIM-2 proteins are fluorescent, detection can be accomplished in real time and in live cells, e.g., by video microscopy. Alternatively, the STIM-2 distribution patterns can be detected in fixed cells. For example, cells expressing a detectible STIM-2 protein can be exposed to an agent or other stimulus for different time periods (e.g., at about 10 s, 20 s, 30 s, 60 s, 90 s, 120 s, 150 s, 180 s, or more, or on the order of several minutes to hours). At the end of the time periods, the cells can be fixed according to a suitable method known in the art (e.g., using a fixative such as paraformaldehye, methanol, or the like). The STIM-2 (and/or STIM-1) distribution patterns can then be detected by detection of the detectable domain.

Where the detectable domain is a fluorescent polypeptide, methods of measuring and/or monitoring fluorescence are well known in the art. Both qualitative assessments (positive/negative) and quantitative assessments (e.g., comparative degree of fluorescence) may be provided by the present methods. Brightness can be measured using any known method, including, but not limited to, visual screening, spectrophotometry, spectrofluorometry, fluorescent microscopy (e.g., confocal microscopy), etc. In some embodiments, monitoring of fluorescent biosensor polypeptides includes the use of an automated imaging system such as an Axon ImageXpress 5000, which can optionally be equipped with a live cell imaging chamber. Other suitable imaging systems include, but are not limited to, BD Biosciences (Pathway HT); Cellomics (ArrayScan V); Amersham (IN Cell Analyzer 1000; IN Cell Analyzer 3000); Molecular Devices (Discovery-1, Discovery-TMA, ImageXpress), and the like. In general, the best quality images may be obtained by focusing the microscope at the bottom of the cell on a support (e.g., the bottom of the cell contained in a microtiter plate).

In embodiments involving use of fixed cells, the cells can be examined at any appropriate time after fixing, preferably at a time after fixing in which the detectable signal from the detectable domain of the STIM-2 protein can be readily detected.

### Nucleic Acids

The subject invention also provides nucleic acid compositions encoding the detectible STIM-2 mutant and wild-type proteins described herein, particularly nucleic acids encoding their respective precursor forms, which precursor forms include a signal peptide. Nucleic acid compositions of particular interest comprise a sequence of DNA having an open reading frame that encodes a calcium biosensor polypeptide of the subject invention and is capable, under appropriate conditions, of being expressed as a protein according to the subject invention.

The subject nucleic acids may be present in an appropriate vector for extrachromosomal maintenance or for integration into a host genome, as described in greater detail below. Preferably the subject nucleic acids are provided for stable maintenance in a host cell in which assays are to be conducted, e.g., as a genomic integrant.

The subject polynucleotides and constructs can be generated by a number of different protocols known to those of skill in the art. Appropriate polynucleotide constructs are purified using standard recombinant DNA techniques as described in, for example, Sambrook et al., Molecular Cloning: A Laboratory Manual, 2nd Ed., (1989) Cold Spring Harbor Press, Cold Spring Harbor, N.Y., and under current regulations described in United States Dept. of HHS, National Institute of Health (NIH) Guidelines for Recombinant DNA Research.

Also provided are constructs comprising the subject nucleic acids inserted into a vector, where such constructs may be used for a number of different applications, including propagation, protein production, etc. In some embodiments, the vector (e.g., a plasmid) will contain nucleic acid having a coding sequence for a detectible STIM-2 protein (either wild type or mutant), preferably including a coding sequence of a signal polypeptide positioned at a 5′ end of the coding sequence to facilitate appropriate processing and insertion of the encoded detectible STIM-2 mutant or wild-type protein in host cell membranes. In embodiments in which both a wild-type and mutant detectible STIM-2 protein are co-expressed in the same host cell, a single vector may be employed to express both proteins or, alternatively, two independent vectors encoding each may be used.

Viral and non-viral vectors may be prepared and used, including plasmids, which provide for replication of STIM-2-encoding DNA and/or expression in a host cell. The choice of vector will depend on the type of cell in which propagation is desired and the purpose of propagation. Certain vectors are useful for amplifying and making large amounts of the desired DNA sequence. Other vectors are suitable for expression in cells, particularly stable expression, in culture. The choice of appropriate vector is well within the skill of the art. Many such vectors are available commercially. Methods for production of such vectors are well known in the art.

Also provided are expression cassettes or systems that find use in, among other applications, the synthesis of the subject proteins. For expression, the gene product encoded by a polynucleotide of the invention is expressed in any suitable host cell in which the detection of the state of basal calcium level regulation is desired, e.g., eukaryotic cells, including mammalian, insect, amphibian and avian cells, and the like. In the expression vector, a subject polynucleotide is operably linked to a regulatory sequence as appropriate to obtain the desired expression properties. These regulatory sequences can include promoters (attached either at the 5′ end of the sense strand or at the 3′ end of the antisense strand), enhancers, terminators, operators, repressors, and inducers. The promoters can be regulated (e.g., inducible) or constitutive. In some situations it may be desirable to use conditionally active promoters, such as tissue-specific or developmental stage-specific promoters. These are linked to the desired nucleotide sequence using the techniques described above for linkage to vectors. Any techniques known in the art can be used. The expression vector will provide a transcriptional and translational initiation region, which may be inducible or constitutive, where the coding region is operably linked under the transcriptional control of the transcriptional initiation region, and a transcriptional and translational termination region. These control regions may be native to the subject species from which the subject nucleic acid is obtained, or may be derived from exogenous sources.

Eukaryotic promoters suitable for use include, but are not limited to, the following: the promoter of the mouse metallothionein I gene sequence (Hamer et al., J. Mol. Appl. Gen. 1:273-288, 1982); the TK promoter of Herpes virus (McKnight, Cell 31:355-365, 1982); the SV40 early promoter (Benoist et al., Nature (London) 290:304-310, 1981); the yeast gall gene sequence promoter (Johnston et al., Proc. Natl. Acad. Sci. (USA) 79:6971-6975, 1982); Silver et al., Proc. Natl. Acad. Sci. (USA) 81:5951-59SS, 1984), the CMV promoter, the EF-1 promoter, Ecdysone-responsive promoter(s), tetracycline-responsive promoter, and the like.

Promoters may be, furthermore, either constitutive or regulatable. Inducible elements are DNA sequence elements that act in conjunction with promoters and may bind either repressors (e.g. lacO/LAC Iq repressor system in E. coli) or inducers (e.g. gal1/GAL4 inducer system in yeast). In such cases, transcription is virtually “shut off” until the promoter is derepressed or induced, at which point transcription is “turned-on.”

Expression vectors generally have convenient restriction sites located near the promoter sequence to provide for the insertion of nucleic acid sequences encoding heterologous proteins. A selectable marker operative in the expression host may be present. Expression vectors may be used for, among other things, the screening methods described in greater detail below. Alternatively, expression vectors can take advantage of recombination systems (e.g., Cre-lox, att sites, and the like) to provide for manipulation of vector components. Exemplary systems include the Creator™ (Clontech) and Gateway™ (Invitrogen) systems.

Expression cassettes may be prepared comprising a transcription initiation region, the gene or fragment thereof, and a transcriptional termination region. After introduction of the DNA, the cells containing the construct may be selected by means of a selectable marker, the cells expanded and then used for expression.

The above described vector systems may be employed with prokaryotes or eukaryotes in accordance with conventional ways. Generally, it is desirable to express the gene in eukaryotic cells, particularly mammalian cells, where the expressed protein are provided in association with a membrane of a sequestered calcium store, e.g., a membrane of an endoplasmic reticulum. Expression in bacterial cells may be desired where purification of the protein may be of interest (e.g., for production of antibodies, particularly monoclonal antibodies and antigen-binding fragments thereof, e.g., to provide a reagent for detection of a detectable domain of the STIM-2 protein, which may be endogenous or heterologous to the STIM polypeptide backbone).

When any of the above host cells, or other appropriate host cells or organisms, are used to replicate and/or express the polynucleotides or nucleic acids of the invention, the resulting replicated nucleic acid, RNA, expressed protein or polypeptide, is within the scope of the invention as a product of the host cell or organism.

The subject nucleic acids may be mutated in various ways known in the art to generate targeted changes in the sequence of the encoded protein, properties of the encoded protein, including fluorescent properties of the encoded protein, etc. The DNA sequence or protein product of such a mutation will usually be substantially similar to the sequences provided herein, e.g. will differ by at least one nucleotide or amino acid, respectively, and may differ by at least two but not more than about ten nucleotides or amino acids. The sequence changes may be substitutions, insertions, deletions, or a combination thereof. Techniques for in vitro mutagenesis, including site specific mutagenesis of cloned polynucleotides are known. Nucleic acids that differ in nucleotide sequence but encode the same amino acid sequence due to the degeneracy of the genetic code are also contemplated, and are referred to herein as “degenerate variants”. In some embodiments where the STIM-2 protein-encoding nucleic acids are of a different origin than the host cell in which the construct is to be expressed, it may be desirable to provide for selection of codons for optimal expression in a particular host cell, e.g., to provide a human-optimized nucleic acid that utilizes codons most frequently used in a human cell.

### Recombinant Cells

The invention also features host cells engineered to express a wild-type and/or mutant STIM-2 protein of the invention, as well as kits and methods of the subject invention using such cells. Of particular interest are recombinant host cells that are modified to provide for stable expression of the wild-type and/or mutant STIM-2 proteins the according to the invention.

While use of the methods and compositions of the invention find particular use in mammalian cells, particularly human cells, the wild-type and mutant STIM-2 proteins of the invention can be used in connection with any eukaryotic cells that normally contain a calcium-responsive STIM polypeptide (Williams et al., (2001) Biochem. J., 357, 673-85).

In general, the subject cells are eukaryotic cells that support production of a wild-type and/or mutant STIM-2 protein, according to the invention. Preferably, the cells are such that can be readily propagated in culture and readily manipulated using recombinant techniques. Exemplary cells, include, but are not necessarily limited to, mammalian cells (particularly human cells), such as Jurkat (human T-lymphocyte) E6-1 cells, HeLa cells, and the like. In some embodiments, the cells are obtained from a patient having a defect in the regulation of basal calcium levels. Cells suitable for use in the invention include cell lines (e.g., immortalized cells) as well as primary cells that are amenable to recombinant manipulation.

In general, the cells are generated by introduction of one or more constructs for expression of a wild-type and/or mutant STIM-2 protein of the invention. As described in greater detail above, in some embodiments a single polynucleotide e.g., plasmid, may encode one or more wild-type and/or mutant STIM-2 proteins. In other embodiments, wild-type and mutant STIM-2 protein are provided on separate constructs for introduction into the host cell.

The constructs can be introduced into the host cell by any one of the standard means practiced by one with skill in the art to produce a recombinant cell of the invention. The nucleic acid constructs can be delivered, for example, with cationic lipids (Goddard, et al, Gene Therapy, 4:1231-1236, 1997; Gorman, et al, Gene Therapy 4:983-992, 1997; Chadwick, et al, Gene Therapy 4:937-942, 1997; Gokhale, et al, Gene Therapy 4:1289-1299, 1997; Gao, and Huang, Gene Therapy 2:710-722, 1995, all of which are incorporated by reference herein), using viral vectors (Monahan, et al, Gene Therapy 4:40-49, 1997; Onodera, et al, Blood 91:30-36, 1998, all of which are incorporated by reference herein), by uptake of “naked DNA”, and the like.

In some embodiments, the wild-type and/or mutant STIM-2 protein are introduced into the cell as polynucleotides encoding the wild-type and/or mutant STIM-2 protein for transient expression (e.g., the vector is maintained in an episomal manner by the cell). In other embodiments, one or more expression constructs encoding a wild-type and/or mutant STIM-2 protein can be stably integrated into a cell line. In addition or alternatively, a polynucleotide encoding a wild-type and/or mutant STIM-2 protein can be stably integrated into the cell, while a wild-type and/or mutant STIM-2 protein can be optionally carried on one or more transient expression vectors. For example, a polynucleotide encoding a wild-type STIM-2 protein may be stably integrated in the cell line, a while a polynucleotide encoding mutant STIM-2 protein is carried on a transient expression vector, or vice versa.

### Methods of Using Detectible STIM-2 Polypeptides

As mentioned above, the subject biosensor polypeptides find particular utility in assays designed to monitor basal intracellular calcium levels, which in turn yields information about the cell's physiological state with respect to basal intracellular calcium regulation. In addition, the subject STIM-2 polypeptides find particular utility in screening assays designed to identify an agent (e.g., a gene product or small molecule compound) that modulates the basal intracellular calcium levels of a eukaryotic cell.

Monitoring Basal Intracellular Calcium Stores

Expression of a wild-type detectible STIM-2 protein in a variety of cell lines allows monitoring of the basal intracellular calcium state of a cell. When basal calcium levels are within normal limits, the detectible STIM-2 will be bound to calcium, and will provide a diffuse pattern when detected. When basal calcium levels are below normal limits, the detectible STIM-2 proteins are not bound to calcium and will provide a punctate pattern. One can monitor these changes in basal intracellular calcium level states over time (e.g., over seconds to several minutes, e.g., 30 s, 60 s, 90 s, 120 s, 150 s, 180 s, 210 s, 240 s, 300 s, 360 s, or longer), such that in a series of detections, the cell will proceed from diffuse detectable pattern to punctate detectable pattern, or vice versa.

In certain embodiments, a detectible STIM-1 protein is employed in the basal calcium level monitoring methods of the present invention. Both STIM-1 and STIM-2 play a role in mediating intracellular calcium flux (e.g., calcium signaling in response to activation signals). However, STIM-1 does not play a significant role in regulating basal calcium levels in a cell. Thus, detectible STIM-1 can serve as a control to differentiate induced calcium flux STIM distribution patterns from STIM-2 distribution patterns related to basal calcium level regulation. For example, a punctate distribution of both STIM-1 and STIM-2 indicates a that calcium flux (e.g., calcium signaling) is occurring in a cell whereas a punctate distribution pattern of STIM-2 and a diffuse distribution pattern of STIM-1 indicates low basal intracellular calcium levels.

Identifying Agents that Modulate Intracellular Calcium Store Levels

As noted above, the subject methods can also be used in screening assays designed to identify an agent (e.g., a gene product or small molecule compound) or other stimulus that modulates basal intracellular levels of calcium. In some embodiments, the modulating agent results in alteration of the basal level of calcium in a cell.

In one embodiment, the subject method is carried out by culturing a cell comprising a detectible STIM-2 protein in the presence of a candidate agent, wherein the detectible STIM-2 protein comprises a fluorescent polypeptide as a detectable domain. If the candidate agent modulates basal intracellular calcium levels such that the basal calcium level is increased, then the distribution pattern of the detectible STIM-2 protein will be more punctate. Conversely, if the candidate agent modulates basal intracellular calcium levels such that the basal calcium level is decreased, then the distribution pattern of the detectible STIM-2 protein will be more diffuse. In certain embodiments, the rate of puncta formation in the presence of the candidate agent compared to, for example, the rate of puncta formation in the presence of a known basal calcium modulator can be assessed. Alternatively, the candidate agent can be screened for the ability to modulate the effect of a known basal calcium level modulator. For example, inhibition of, or a decreased rate of, basal calcium level reduction in the presence of the agent can indicate an obstructing effect of the candidate agent upon basal intracellular calcium level reduction depletion by the known basal calcium modulator. The invention can also involve screening to determine whether a known drug modulates basal intracellular levels of calcium.

As noted above, all assays described herein can be conducted with live cells. Alternatively, the cells can be fixed, and detection of detectible STIM-2 distribution patterns assessed. The use of fixed cells finds particular application in candidate agent screening assays. In general, the cells of the invention are contacted with a candidate agent, and, after a desired time period, the cells are fixed and the detectible STIM-2 distribution patterns in the presence the agent (or, as a control, in the absence and/or different concentrations of the agent) detected. In certain embodiments, the distribution pattern of a mutant STIM-2 protein and/or a detectible STIM-1 protein is also determined. For example, cells expressing a detectible STIM-2 protein can be exposed to an agent or other stimulus for different time periods (e.g., at about 10 s, 20 s, 30 s, 60 s, 90 s, 120 s, 150 s, 180 s, or more, or on the order of several minutes to hours). At the end of the desired time periods, the cells can be fixed according to a suitable method known in the art (e.g., using a fixative such as paraformaldehye, methanol, or the like). The STIM-2 distribution patterns can then be detected by detection of the detectable domain.

Use of fixed cells has several advantages. For example, once the cells are fixed, detection of the pattern of STIM-2 distribution is not as time sensitive as in live cells. Detection of STIM-2 distribution using fixed cells can take advantage of detection systems that are not as amenable to use in live cells, e.g., antibody-based detection systems. Also, use of fixed cells in the assays of the invention make the assays very amenable to high throughput, since many different assays can be run in parallel and the results of those assays examined at a later time point.

Assays of the invention make it possible to identify agents (such as a gene product or a compound) which increase or decrease basal calcium levels in a cell. Such agents may be therapeutic candidates for treating pathologic conditions associates with dysregulated basal calcium levels in cells (e.g., conditions in which basal calcium levels in cell are increased or decreased from normal levels for long periods of time).

Generally a plurality of assay mixtures is performed in parallel with different agent concentrations to obtain a differential response to the various concentrations of candidate agent. Typically, one of these concentrations serves as a negative control, i.e. no compound. In a preferred embodiment, a high throughput screening protocol is employed, in which a large number of candidate agents are tested in parallel using a large number of cell populations. By “large number” is at least 10 to 50, usually at least 100, and more usually at least 1000.

Of particular interest in certain embodiments is the use of the subject methods in a high throughput toxicity screening assays. In such high throughput screening (HTS) assays, a plurality of different compound compositions, usually at least 10 different compound compositions, are simultaneously assayed for their activity, if any. Each compound composition in the plurality is assayed for activity by contacting it with a cell comprising the subject biosensor polypeptides and determining the effect of the compound composition on intracellular stored calcium levels.

A variety of different candidate agents may be screened by the above methods. Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Moreover, screening may be directed to known pharmacologically active compounds and chemical analogs thereof, or to new agents with unknown properties such as those created through rational drug design.

The above screening methods may be part of a multi-step screening process of evaluating candidate agents for their efficacy (and safety) in the treatment of conditions associated with dysregulated basal cellular calcium levels. Such conditions include, but are not limited to, Alzheimer disease, endothelial dysfunction, kidney disease, cardiac dysfunction (e.g., hypertrophic cardiomyopathy), Huntington disease, as well as other neurodegenerative and aging related diseases. As such, aspects of the invention include the treating a subject having a condition associated with dysregulated cellular basal calcium levels by administering an effective amount of an agent that modulates cellular STIM-2 activity. Modulating STIM-2 activity includes modulating STIM-2 calcium binding, modulating STIM-2 aggregation, modulating STIM-2 expression level, and modulating STIM-2-mediated calcium transport. For example, in conditions characterized by low basal calcium levels, the agent would increase cellular STIM-2 activity. Conversely, in conditions characterized by high basal calcium levels, the agent would decrease cellular STIM-2 activity.

In multi-step screening processes of the subject invention, a candidate compound or library of compounds is subjected to screening in a second in vivo model, e.g. a mouse model, following screening in the subject cell lines. Following the initial screening in the cell lines of the subject invention, the positive compounds are then screened in non-human mammalian animal models. In addition, a pre in vivo screening step may be employed, in which the compound is first subjected to an in vitro screening assay for its potential as a therapeutic agent in the treatment of disease or condition of interest. Any convenient in vitro screening assay may be employed, where a variety of suitable in vitro screening assays are known to those of skill in the art.

In some embodiments, the subject methods are useful for identifying a endogenous gene product that has an activity in modulating the basal levels of calcium in a cell. Genes that have a beneficial effect on the phenotype when their activity is modulated through mutation encode proteins that represent therapeutic targets for the development of compounds that inhibit the function of the protein. Gene based therapies can be identified by doing traditional enhancer/suppressor analyses in the subject cells. In these analyses, genes in the subject cells are mutated to identify ones that either increase or decrease basal calcium levels. Methods of mutating genes and carrying out enhancer/suppressor analyses are well known to those of skill in the art (Hays, T S et al., Molecular and Cellular Biology (March 1989) 9(3):875-84; Deuring, R; Robertson, B; Prout, M; and Fuller, M T. Mol. Cell. Biol., 1989 9:875-84; Fuller, M T et al., Cell Mot. Cyto. (1989) 14:128-35; Rottgen G, Wagner T, Hinz U Mol. Gen. Genet. 1998 257:442-51). In some embodiments, siRNA is used to disrupt the expression of an endogenous gene to determine whether the endogenous gene had an effect on modulating basal calcium levels.

Automated Screening Methods

The methods of the present invention may be automated to provide convenient, real time, high volume methods of screening compounds for activity in modulation of intracellular calcium stores. Automated methods are designed to detect changes in the pattern of the detected signal (usually fluorescence) of one or more of the detectible STIM-2 polypeptides over time (i.e., comparing the same apparatus before and after exposure to a test sample), or by comparison to a control apparatus, which is not exposed to the test sample, or by comparison to pre-established indicia. Both qualitative assessments (positive/negative) and quantitative assessments (e.g., concentration of test sample required to promote or inhibit calcium store depletion) may be provided by the present automated methods.

An embodiment of the present invention includes an apparatus for detecting changes in basal calcium levels according to the subject methods of the present invention. This apparatus comprises means, such as a fluorescence measurement tool, for measuring change in the pattern of a detectable signal, such as a fluorescence pattern, associated with one or more STIM proteins in a eukaryotic cell in response to a particular candidate agent.

Measurement points may be over time, or among test and control samples. A computer program product controls operation of the measuring means and performs numerical operations relating to the above-described steps. The preferred computer program product comprises a computer readable storage medium having computer-readable program code means embodied in the medium. Hardware suitable for use in such automated apparatus will be apparent to those of skill in the art, and may include computer controllers, automated sample handlers, fluorescence measurement tools, printers and optical displays. The measurement tool may contain one or more photodetectors for measuring the fluorescence signals from samples where fluorescently detectable molecules are utilized. The measurement tool may also contain a computer-controlled stepper motor so that each control and/or test sample can be arranged as an array of samples and automatically and repeatedly positioned opposite a photodetector during the step of measuring fluorescence intensity.

The measurement tool is preferably operatively coupled to a general purpose or application specific computer controller. The controller preferably comprises a computer program produced for controlling operation of the measurement tool and performing numerical operations relating to the above-described steps. The controller may accept set-up and other related data via a file, disk input or data bus. A display and printer may also be provided to visually display the operations performed by the controller. It will be understood by those having skill in the art that the functions performed by the controller may be realized in whole or in part as software modules running on a general purpose computer system. Alternatively, a dedicated stand-alone system with application specific integrated circuits for performing the above described functions and operations may be provided.

### Kits and Systems

Also provided by the subject invention are kits and systems for use in practicing the subject methods, where the subject kits can include elements for making the subject detectible STIM-2 polypeptides, e.g., a construct comprising a vector that includes a coding region for the subject biosensor polypeptides. In some embodiments, the subject kits and systems can include, in separate compartments or containers, one or more of the following: 1) one or more constructs encoding one or more of the STIM-2 proteins (either wild-type of mutant) of the invention; 2) a candidate agent; and 3) a cell containing an expression construct for producing one or more of the detectible STIM-2 proteins of the invention. The components of the kits may be modified commensurate to the disclosure provided above.

The subject kit components are typically present in a suitable storage medium, e.g., buffered solution, typically in a suitable container. In certain embodiments, the kit comprises a plurality of different vectors each encoding the subject protein, where the vectors are designed for expression in different environments and/or under different conditions, e.g., constitutive expression where the vector includes a strong promoter for expression in specific eukaryotic cells, a promoterless vector with a multiple cloning site for custom insertion of a promoter and tailored expression of a biosensor polypeptide, etc.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Methods and Materials

The following methods and materials are used in the examples below.

Cell Transfection, Plasmids, and Reagents

Stim1 synthetic siRNA was purchased from Ambion. Stim2 synthetic siRNA was purchased from Dharmacon. Diced siRNA against GL3 luciferase was used as a transfection control. HeLa, HUVEC, and HEK293T cells were purchased from ATCC. DNA plasmids were transfected with Fugene 6 reagent (Roche). siRNA was transfected with Gene Silencer reagent (Gene Therapy Systems, San Diego, Calif.) for HeLa, Lipofectamine 2000 reagent (Invitrogen) for HEK293T, and Lipofectin reagent (Invitrogen) for HUVEC. Full-length human Stim1 and Stim2 cDNA were isolated by PCR, sequenced, and cloned in to pDS_XB-YFP vector (ATCC). Enhanced yellow fluorescent protein (YFP) or cyan fluorescent protein (CFP) (Clontech, Palo Alto) was inserted immediately downstream of the predicted signal-peptide region of human Stim1 and Stim2. The YFP-conjugated EF-hand mutants of Stim1, Stim1 EF(D76A), and Stim2, Stim2 EF(D68A) were made by site-directed mutagenesis with the QuikChange Site-Directed Mutagenesis Kit (Stratagene). Thapsigargin (Invitrogen) and ionomycin (Sigma) were used at 1 μM.

siRNA Library of Signaling Proteins

The synthesis of the Dicer generated siRNA library was described previously (Liou et al., 2005). In short, 2400 human signaling-related proteins were selected from the NCBI (RefSeq database) on the basis of the presence of signaling domains, such as protein kinase, SH2, SAM, EF, and PH domains, as well as by text searches of signaling-related terms. Gene-specific primers for the selected signaling proteins were designed with an in-house primer program and were used to generate ˜600 bp cDNA fragments immediately upstream of the stop codon of each mRNA by PCR. An additional set of nested primers was designed to add T7 promoters at both ends of the final cDNA fragment. Nested PCR products were subjected to in vitro transcription, in vitro dicing, and purification to produce siRNA as described previously (Myers et al., 2003). The siRNA signaling set was sorted according to the NCBI RefSeq Protein accession number and was stored in 24 96-well plates.

Ca²⁺ Measurements

HeLa cells were loaded with 0.5 μM Fura-2-AM in extracellular buffer (125 mM NaCl, 5 mM KCl, 1.5 mM MgCl2, 20 mM HEPES, 10 mM glucose, and 0.1 mM CaCl2 or 11.8 mM CaCl2 [pH 7.4]) for 30 min at room temperature. Fura-2 fluorescence was measured by illuminating the cells with an alternating 340/380 nm light and measuring fluorescence intensity at 510 nm. Intracellular Ca²⁺ concentration was computed from the ratio of fluorescence intensity for excitation at 340 and 380 nm. For the RNAi screen, the values for each well of the 384 well plate have been subject to two normalization steps. First, regional background subtraction was performed between neighboring wells to correct for trends across the well plate. Finally, the values are presented are fold standard deviations from the median of the screen. For all other figures, Fura-2 ratio was calibrated using:

[Ca²⁺]=Kd*Q*(R−Rmin)/(Rmax−R)

where Kd=0.14 uM Rmax was determined by measuring the maximal attainable fluorescence intensity in cells treated with ionomycin in high external Ca²⁺ (Rmax=6.06). Rmin and Q values are dependent on a cells autofluorescence, unspecific Fura-2 binding to cellular components as well as on Fura-2 sequestration inside cells. We adjusted them for HeLa cells so that the average basal Ca²⁺ level was 50 nM in calibration experiments (Rmin=0.217 and Q=9.71). The same Rmax, Rmin and Q values were used for all experiments shown.

For Ca²⁺ add-back experiments, 3 mM EGTA was added together with thapsigargin to remove extracellular Ca²⁺, and extracellular Ca²⁺ was changed to a final Ca²⁺ concentration of 0.75 mM 15 minutes later. Imaging-based single cell Ca²⁺ measurements were performed with a 4× objective on an automated fluorescent microscope (ImageXpress 5000A, Molecular Devices). Fluorescence intensities of single cells were calculated using Matlab 7.1 software.

Extracellular Ca²⁺ manipulation

For the RNAi screen, low Ca²⁺ conditions were created by replacing normal media (Dulbecco's Modified Eagle's Medium (DMEM), 10% fetal bovine serum, penicillin, streptomycin, glutamate) with media made using DMEM without Ca²⁺ (Invitrogen). For other experiments, low Ca²⁺ media was created by adding 2 mM EGTA to normal media. High extacellular Ca²⁺ media was made by adding 10 mM Ca²⁺ to normal media.

Confocal Imaging

Confocal imaging was performed using a Zeiss spinning disk confocal. Cells were imaged in extracellar buffer as described in the Ca²⁺ measurements section.

Puncta Analysis

Puncta analysis for Stim1 and Stim2 translocation were performed by applying a bandpass filter and then taking the mean of the square of the pixels in the resulting image. Each time-series of values was then linearly transformed to range between 0 and 1.

### Example 1

### Design of a Sensitized siRNA Screen to Identify Regulators of Basal Ca²⁺ Concentration

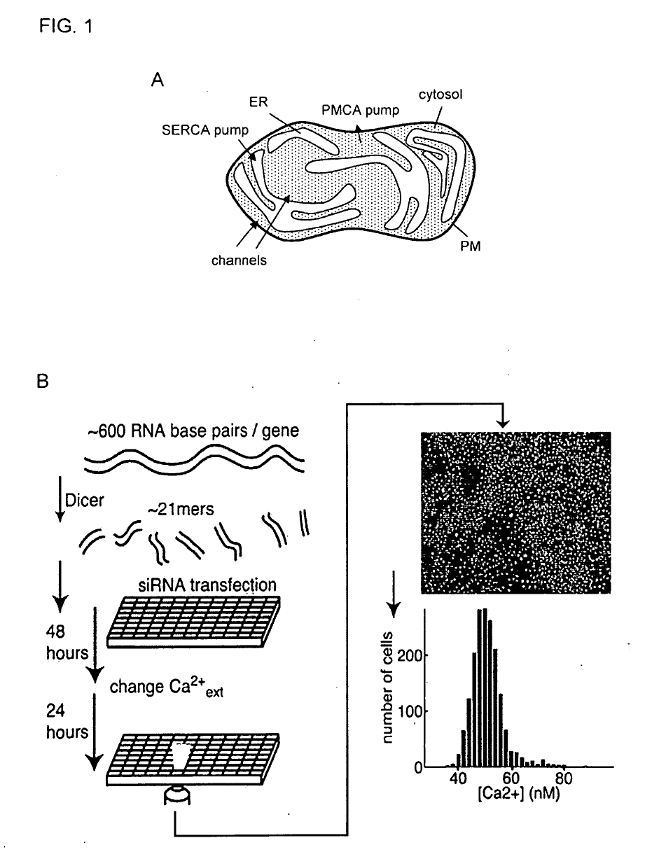

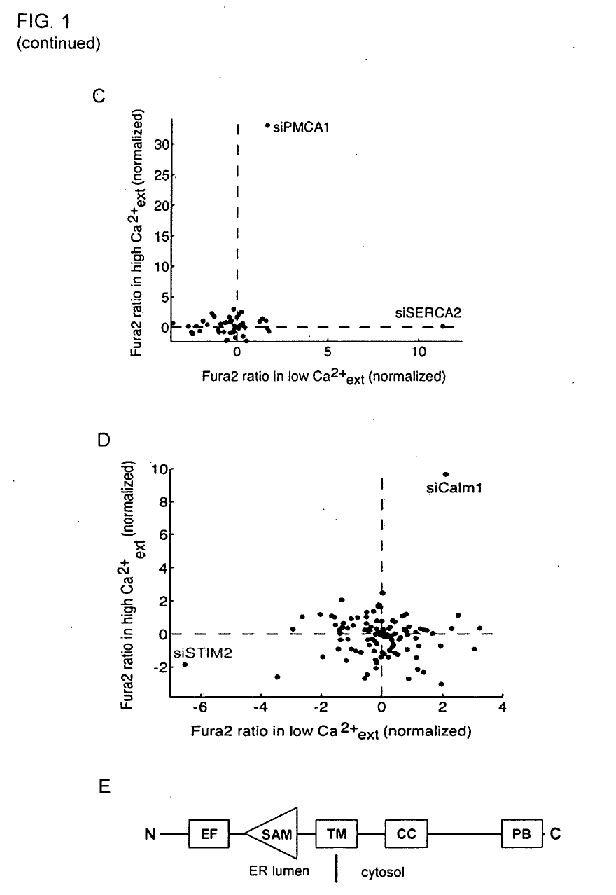

We developed a siRNA screening protocol to identify human genes that regulate basal cytosolic Ca²⁺ concentration. HeLa cells were sensitized by subjecting their Ca²⁺ signaling system to two opposing pressures: 24 hours in high extracellular Ca²⁺ (+10 mM) and, in a separate experiment, 24 hours in low extracelluar Ca²⁺ (˜0.1 mM). This strategy was devised to push the Ca²⁺ homeostatic control system towards its limits so that the effect of the RNAi perturbations could be more readily observed. We used the ratiometric Ca²⁺ dicator Fura-2 (Grynkiewicz et al., 1985) and an automated fluorescence imaging system to measure siRNA-mediated differences in basal Ca²⁺ concentration. Approximately 1000 individual cells in each well of a 384-well plate were analyzed and the mean single cell Fura-2 ratio computed for each well (FIG. 1B).

We tested the usefulness of our strategy by making a Dicer-generated Ca²⁺ siRNA set that included known and putative Ca²⁺ pumps, channels and exchangers (FIG. 1B). Among this set, the strongest hit in the high external Ca²⁺ condition was the plasma membrane Ca²⁺-ATPase gene PMCA1 (FIG. 1C; Table 1 for a list of results). The marked effect of Pmca1 knockdown is consistent with a role of plasma membrane Ca²⁺ pumps as a main avenue for Ca²⁺ extrusion out of the cell (Guerini et al., 2005). The strongest hit in the low external Ca²⁺ condition was the endoplasmic-reticulum Ca²⁺ pump gene SERCA2 (FIG. 1C), which pumps Ca²⁺ from the cytosol into the ER (Strehler et al., 2007). This effect of knocking down an ER Ca²⁺ pump was also expected given that thapsigargin, an inhibitor of Serca pumps, is known to trigger store-depletion triggered Ca²⁺ influx and persistent increases in cytosolic Ca²⁺ concentration (Thastrup, 1990). It is reasonable that Serca2 was not hit in the high external Ca²⁺ condition because the increased cytosolic Ca²⁺ level likely compensated for the lower concentration of Serca pumps. These test measurements demonstrated that our assay system is useful for identifying different types of regulators of basal Ca²⁺ concentration.

TABLE 1
Sensitized Screen Results of known and putative
Ca²⁺ pumps, channels and exchangers

| | low Ca²⁺ ext | high Ca²⁺ ext |
|---|---|---|
| Gene | condition | condition |
| ATP2A1 | −0.39289 | −0.9418 |
| ATP2A2 | 11.3003 | 0.142401 |
| ATP2A3 | −0.35855 | 1.1669 |
| ATP2B1 | 1.641 | 33.0733 |
| ATP2B2 | −0.57369 | −2.2089 |
| ATP2B3 | −0.12276 | −0.035867 |
| ATP2B4 | 0.33795 | −1.5009 |
| CACNA1A | −0.58811 | 0.79601 |
| CACNA1B | 0.17067 | 0.36025 |
| CACNA1C | −0.65638 | 0.73057 |
| CACNA1D | −0.17218 | 0.2902 |
| CACNA1E | −1.8773 | 1.03 |
| CACNA1F | 0.088143 | 2.1226 |
| CACNA1G | 0.086275 | −0.19972 |
| CACNA1H | 1.3862 | 1.4276 |
| CACNA1I | −0.9814 | −0.79492 |
| CACNA1S | −2.4325 | −1.098 |
| ITPKA | 1.7505 | −0.64677 |
| ITPKB | 1.5912 | 1.0806 |
| ITPR1 | 1.6406 | −0.12037 |
| ITPR2 | 1.2437 | 0.9335 |
| ITPR3 | 0.51593 | −2.2621 |
| PSEN1 | −2.2494 | 0.18205 |
| PSEN2 | −0.11029 | −1.7258 |
| SLC8A1 | −2.6531 | 0.15688 |
| SLC8A2 | 0.063456 | 0.049516 |
| SLC8A3 | −2.4966 | −0.81094 |
| TRPA1 | −2.0646 | −0.63463 |
| TRPC1 | 0.31108 | 0.63618 |
| TRPC3 | −1.3776 | 2.274 |
| TRPC4 | 0.32254 | 0.58842 |
| TRPC5 | −0.21547 | −0.79262 |
| TRPC6 | −0.15239 | −0.66884 |
| TRPC7 | −1.2503 | 1.7674 |
| TRPM1 | −0.55947 | −2.1096 |
| TRPM2 | −1.6407 | 0.45256 |
| TRPM3 | −0.31696 | 1.0616 |
| TRPM4 | −1.0307 | −0.45337 |
| TRPM5 | −0.89363 | 0.5181 |
| TRPM6 | −0.42829 | 1.6638 |
| TRPM7 | −3.5 | 0.67418 |
| TRPM8 | −0.19636 | −0.49052 |
| TRPV1 | 0.43127 | −0.058567 |
| TRPV2 | −0.019 | 1.6494 |
| TRPV3 | −0.72963 | −0.76843 |
| TRPV4 | −0.36298 | 0.85333 |
| TRPV5 | 0.23015 | 2.4792 |
| TRPV6 | −0.19674 | 2.9569 |

Units are fold standard deviations from median Fura-2 ratio.
Gene names are based on the NCBI nomenclature.

D a ax, high:a+eyxt ATP:2 μl tl 9±9418ATP2A2′ I O 1441

### Example 2

### Identification of Stim2 as a Primary Regulator of Basal Ca²⁺ Concentration

We then used the same assay system to screen the Dicer-generated siRNA library targeting the human signaling proteome (FIG. 1B). 2400 signaling genes were individually knocked down and experiments were performed in duplicate using both the high and low external Ca²⁺ conditions. From this initial screen, we selected statistically significant positive and negative regulators of basal Ca²⁺ concentration and remade Dicer-generated siRNA constructs for the top 112 putative hits. We then re-screened these siRNAs in triplicate and found that the siRNAs targeting STIM2 and calmodulin 1 (CALM1) were the strongest positive and negative regulators, respectively (FIG. 1D). Table 2 lists the statistically significant results from these two high and low Ca²⁺ screens.

TABLE 2
Statistically significant results from high and low Ca²⁺ screens

| | low Ca²⁺ | high Ca²⁺ |
|---|---|---|
| Gene | ext condition | ext condition |
| ACTR3 | −2.6427 | 1.032 |
| AKAP7 | 3.0534 | −0.91885 |
| CALM1 | 2.0908 | 9.6558 |
| CLMN | −0.47456 | −2.4542 |
| FLJ45651 | 1.1813 | −2.1474 |
| FNBP2 | −0.55684 | −2.683 |
| HAPIP | −2.9538 | 0.29199 |
| KIAA0007 | −1.3395 | 2.0522 |
| LOC124685 | 0.031095 | 2.4768 |
| LOC389246 | 2.5197 | 1.12 |
| LOC55971 | 3.2384 | 0.34667 |
| PIP5K1A | −2.0373 | 1.1826 |
| PPEF1 | 0.87681 | −2.7225 |
| PPP1R12A | 2.3008 | 0.33042 |
| PRKAR1A | 1.9521 | −3.0284 |
| RAC1 | −3.4616 | −2.6044 |
| SEC23IP | 1.3801 | −2.3268 |
| SSH1 | −0.18364 | −2.0778 |
| Stim2 | −6.5051 | −1.8468 |

Units are fold standard deviations from median Fura-2 ratio.
Gene names are based on the NCBI nomenclature.

Consistent with the known function of calmodulin to bind and activate PM Ca²⁺ pumps, Calm1 knockdown had a similar relative effect as Pmca1 knockdown for both sensitized conditions. Surprisingly, however, the STIM2 gene product was the number one hit in the low Ca²⁺ ext condition. We were particularly intrigued by this finding since Stim2 shares 47% homology to the recently identified ER-Ca²⁺ sensor Stim1 which functions at the center of a signaling pathway that links receptor-mediated release of ER Ca²⁺ to the opening of plasma membrane Ca²⁺ channels (Roos et al., 2005; Liou et al., 2005). Conflicting results have been reported about a possible role of Stim2 in negatively regulating Stim1 (Soboloff et al., 2006a) or positively regulating storeoperated Ca²⁺ influx (Soboloff et al., 2006b; Liou et al., 2005). Stim2 is a multidomain protein that is at least partially localized to the ER, interfacing both the lumen of the ER and the cytosol (FIG. 1E) (Liou et al., 2005; Dziadek and Johnstone, 2007). We focused our subsequent studies on the role of Stim2 in regulating basal Ca²⁺ homeostasis.

### Example 3

### Stim2 Knockdown Selectively Lowers Basal Cytosolic and ER Ca²⁺ Concentrations

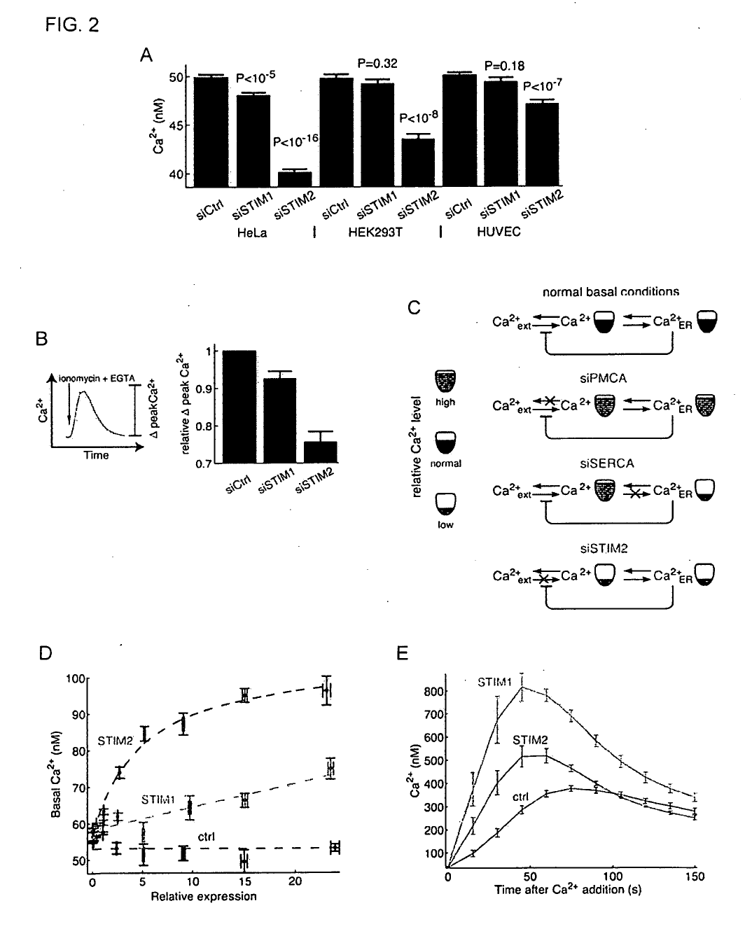

We used synthetic STIM2 siRNA to investigate the effect of Stim2 knockdown on basal Ca²⁺ concentration under normal external Ca²⁺ concentrations (1.5 mM). We also compared the effect of a knockdown of Stim2 to that of its isoform Stim1. Consistent with a unique role of Stim2 in regulating basal Ca²⁺, Stim2 but not Stim1 knockdown significantly lowered basal cytosolic Ca²⁺ in HeLa, HUVEC and HEK293T cells (FIG. 2A).

We also determined the consequence of Stim2 or Stim1 knockdown on the basal Ca²⁺ level in ER stores. We tested ER Ca²⁺ levels by addition of the membrane permeant Ca²⁺ ionophore ionomycin together with external application of the Ca²⁺ chelator EGTA and by quantifying the induced cytosolic Ca²⁺ peak (FIG. 2B). Ionomycin is known to rapidly release Ca²⁺ from ER Ca²⁺ stores and external EGTA was included to prevent Ca²⁺ influx from outside the cell. It is therefore expected that the measured relative amplitude of the induced Ca²⁺ peak (Δpeak Ca²⁺) directly reflects the loading level of ER Ca²⁺ stores. Using this approach, Stim2 knockdown led to a marked decrease in ER Ca²⁺ levels while Stim1 knockdown had a smaller effect. Thus, knockdown of Stim2 reduces both basal cytosolic and ER Ca²⁺ concentrations. The effect of Pmca1, Serca2, and Stim2 knockdowns on basal cytosolic Ca²⁺ levels can be parsimoniously explained by a simple model relating the extracellular, cytosolic, and ER Ca²⁺ pools (FIG. 2C). In unstimulated cells with intact pumps and channels, long-term changes in cytosolic Ca²⁺ concentration are expected to be paralleled by changes in ER Ca²⁺ concentration. Pmca knockdown attenuates export of Ca²⁺ from the cell and would therefore result in an increase in cytosolic and ER Ca²⁺ concentrations. Serca knockdown lowers ER Ca²⁺ levels, thereby activating store-operated Ca²⁺ influx which increases cytosolic Ca²⁺ concentration. In contrast, the finding that Stim2 knockdown lowers Ca⁺ concentration both in the cytosol and ER suggests that it has a role as a regulator of basal Ca²⁺-influx.

### Example 4

### Stim2 and Stim1 have Distinct Roles in Regulating Basal Versus Storeoperated Ca²⁺ Influx

We next used over-expression of YFP-Stim1 and YFP-Stim2 to better understand differential roles of the two isoforms in regulating Ca²⁺ influx. We measured in thousands of cells the single cell concentration of expressed YFPStim1 or YFP-Stim2 constructs as well as single cell Ca²⁺ concentration. The YFP intensities were normalized using the autofluorescence of untransfected cells as a reference and then binned according to different ranges of expression. Transient expression (9 hour) of YFP-Stim2 increased basal Ca²⁺ levels much more than expression of the same concentration of YFP-Stim1 (FIG. 2D). YFP expression is shown as a control. This provides further support for a primary role of Stim2 but not of Stim1 in regulating basal Ca²⁺ concentration. We also found that Stim2 increased Ca²⁺ influx under store depleted conditions, albeit less than Stim1 (FIG. 2E), suggesting that both Stim1 and Stim2 provide a link from ER-Ca²⁺ concentration to Ca²⁺-influx. This led to the working model that Stim2 serves as the primary regulator for basal Ca²⁺-influx while Stim1 and Stim2 both trigger Ca²⁺ influx following receptor-mediated ER store-depletion. In order to separate these two processes, we are abbreviating the basal Ca²⁺ influx regulated by Stim2 as B-SOC, for basal store-operated Ca²⁺ influx, and the receptor-triggered Ca²⁺-influx mediated by Stim1 or Stim2 as R-SOC, for receptor-mediated store-operated Ca²⁺ influx.

### Example 5

### ER Ca²⁺ Store Depletion Triggers Stim2 Translocation to ER-PM Junctions

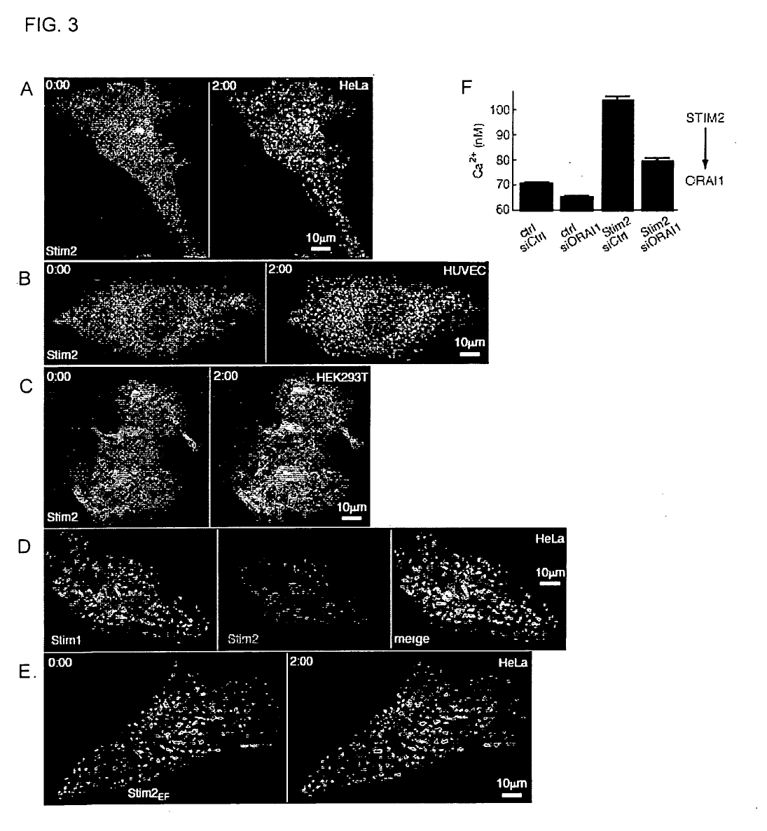

We employed the YFP tagged Stim2 construct to investigate its localization and activation mechanism. Cells with submaximal YFP-Stim2 expression were selected to minimize expression artifacts. Similar to the previously characterized Stim1, YFP-Stim2 expression initially showed a mostly ER-like distribution and ER Ca²⁺ store depletion by thapsigargin led to a rapid translocation of YFPStim2 to PM localized puncta (FIG. 3A). We observed the same YFP-Stim2 puncta formation in HeLa, Huvec, and HEK293T cell lines (FIGS. 3A-C). We then compared Stim1 and Stim2 localization by co-transfection of CFP-Stim1 and YFP-Stim2 followed by thapsigargin-induced depletion of ER Ca²⁺ stores. CFP-Stim1 and YFP-Stim2 were co-localized in the same puncta that have previously been characterized as ER-PM junction sites (FIG. 3D) (Liou et al., 2005, Wu et al., 2006). Finally, we tested whether the punctuate localization of Stim2 is driven by Ca²⁺ binding to its EF-hand by creating an EF hand mutant of Stim2 that disrupts the EF-hand Ca²⁺ binding site (Asp 68 to Ala). The Stim2 EF-hand mutant was prelocalized to ER-PM junctions and did not change distribution in response to thapsigargin (FIG. 3E). This argues that Stim1 and Stim2 make use of a common regulatory mechanism that involves Ca²⁺ dissociation, oligomerization and translocation in order to signal from the lumen of the ER to the ER-PM junction sites (Liou et al., 2007).

Given these similarities in Stim1 and Stim2 activation, we tested whether the effect of Stim2 on B-SOC and basal Ca²⁺ concentration is mediated by the PM Ca²⁺ channel Orai1, as has been shown for R-SOC in the case of Stim1 (Feske et al., 2006; Peinelt et al. 2006; Luik et al., 2006; Yeromin et al., 2006; Mercer et al., 2006). To investigate the potential role of Orai1 in Stim2 signaling, we expressed Stim2 in cells in which we had knocked down Orai1 expression using siRNA preincubation (48 hours). Orai1 knockdown abrogated the effect of Stim2 expression on basal Ca²⁺ by more than 50% (FIG. 3F). This argues that both B-SOC and R-SOC rely in these cells mostly on the opening of Orai1 Ca²⁺ channels to trigger Ca²⁺ influx (FIG. 3F).

### Example 6

### Selective Translocation of Stim2 in Response to Small Reductions in ER Ca²⁺ Concentration

Our imaging and functional studies suggested that several features of the molecular regulation of Stim2 and Stim1 are conserved. A plausible mechanism explaining why they differentially regulate basal Ca²⁺ is a different sensitivity of Stim1 and Stim2 for ER Ca²⁺ concentration. According to this hypothesis, Stim2 is already active at basal ER Ca²⁺ concentrations and becomes more active by small reductions in ER Ca²⁺, while Stim1 requires much larger receptor-triggered reductions in ER Ca²⁺ to be engaged.

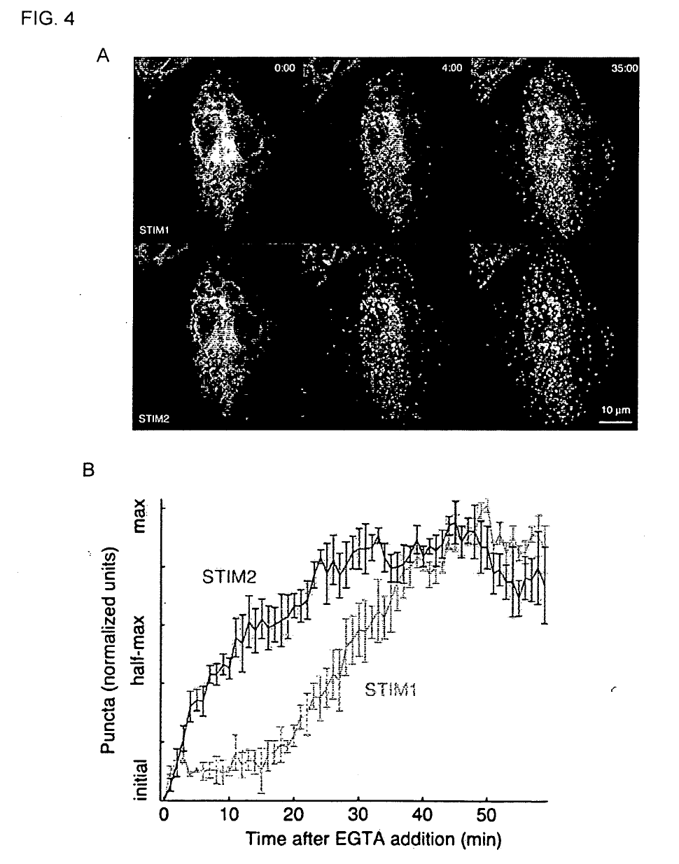

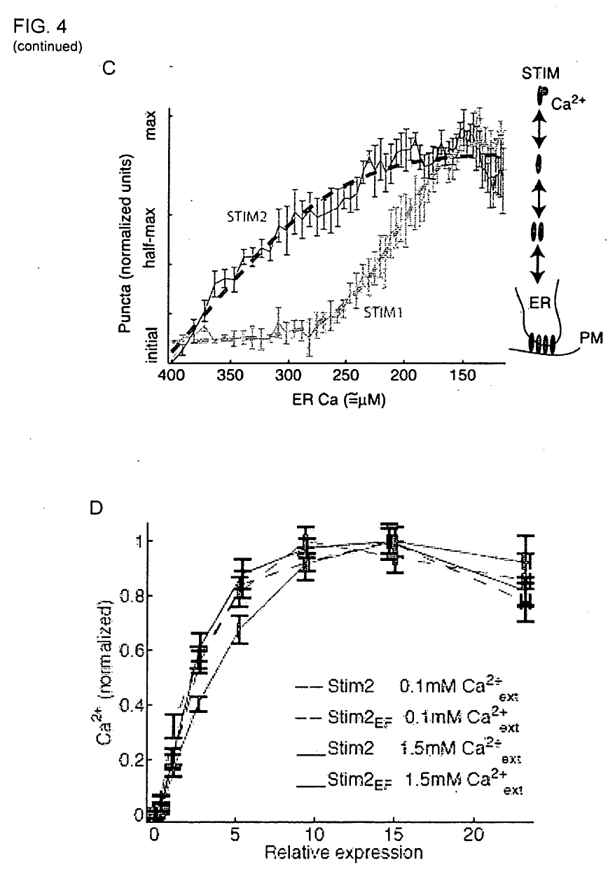

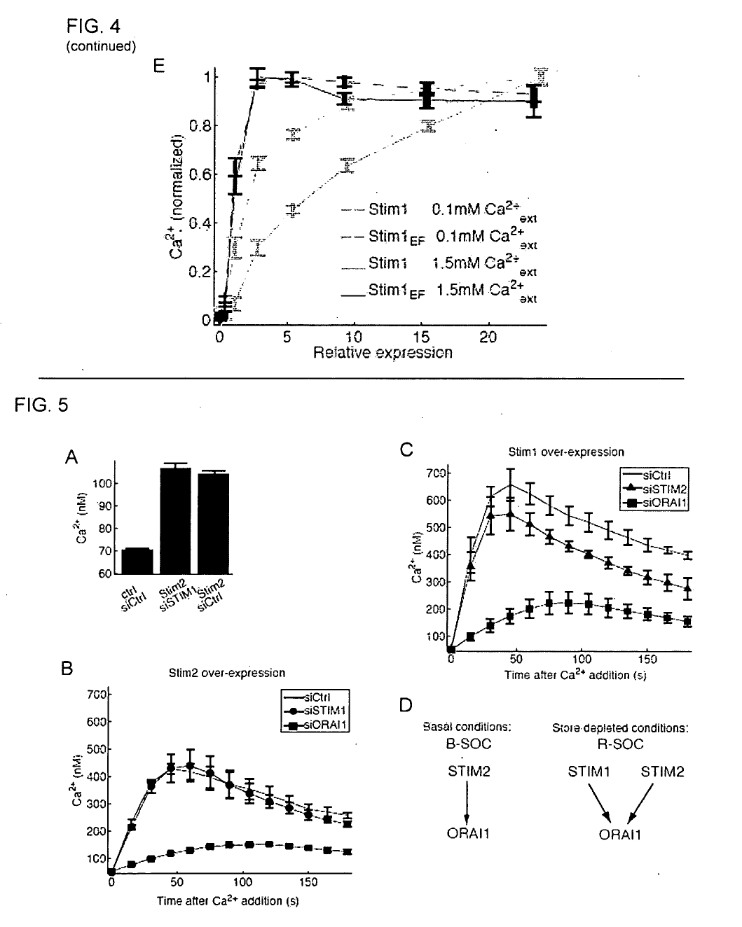

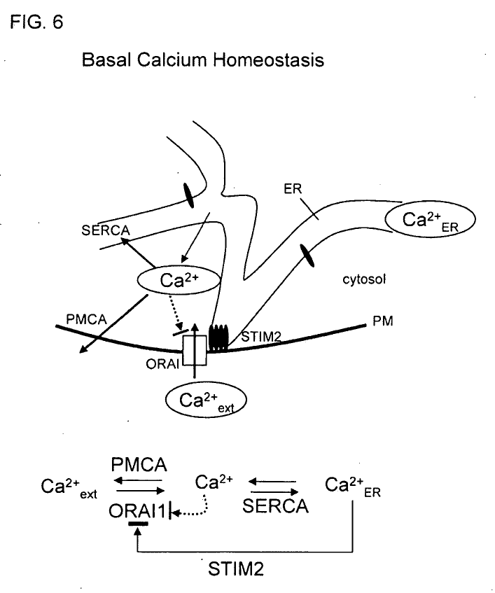

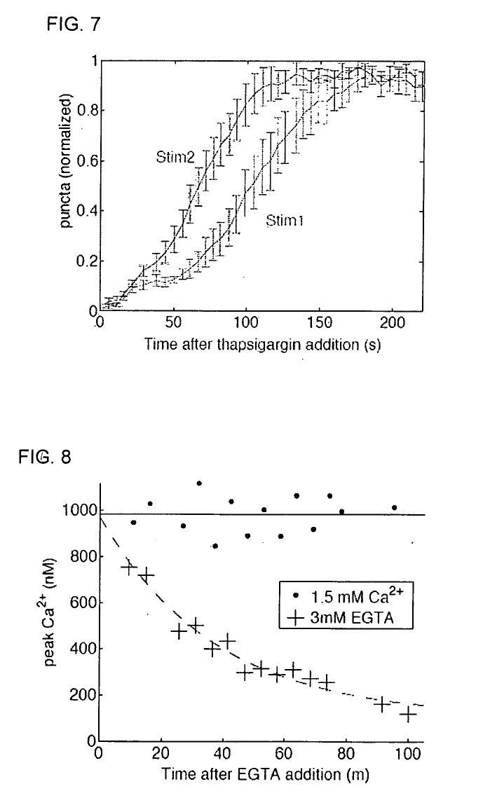

To test this hypothesis, HeLa cells were cotransfected with CFP-Stim1 and YFP-Stim2 and then subjected to slow store depletion by external EGTA addition. Strikingly, YFP-Stim2 started to form puncta at ER-PM junctions shortly after external EGTA addition (time to reach half-maximal puncta˜=10 minutes), while Stim1 puncta formation took much longer (time to half-max puncta˜=30 minutes) (FIG. 4A-B). Stim2 also formed puncta before Stim1 when ER stores were more rapidly depleted by thapsigargin (FIG. 8). These experiments strongly suggest that Stim2 has a lower affinity for Ca²⁺ and therefore translocates to ER-PM junctions and activates Ca²⁺ influx at higher ER Ca²⁺ levels compared to Stim1.

In order to estimate the relative ER Ca²⁺ levels and the degree of cooperativity for translocation of Stim1 and Stim2, we used ionomycin triggered Ca²⁺ release to estimate the remaining Ca²⁺ in ER stores at different time points after EGTA addition (FIG. 8). Assuming that the basal ER Ca²⁺ concentration is approximately 400 μM (Brini et al., 1999; Yu and Hinkle, 2000), we plotted the degree of Stim1 and Stim2 translocation as a function of ER Ca²⁺ levels (FIG. 4C) and fit the ER-Ca²⁺ dependence of translocation to a cooperative activation model. Since the observed puncta formation has been shown to involve sequential Ca²⁺ dissociation, oligomerization, and translocation steps (Liou et al., 2007), our STIM activation model computes the overall cooperativity of the multi-step process (FIG. 4C):

STIM Puncta=α−β*Ca²⁺ERN/(Ca²⁺ERN+EC50N)

Where N is the cooperativity of the overall process, EC50 is the Ca²⁺ concentration for half-maximal translocation and α and β are normalization constants.

A best fit of this model was obtained for EC50 for Stim2 and Stim1 of 406 and 210 μM, respectively, and N=5 and 8 for the cooperativity of Stim2 and Stim1, respectively. This is consistent with a predicted lower Ca²⁺ sensitivity of Stim2 compared to Stim1. An important finding was that Stim1 translocation is regulated with high cooperativity in relation to the ER Ca²⁺ concentration. This cooperativity, combined with its higher affinity for Ca²⁺, can explain how Stim1 is kept effectively inactive at basal ER Ca²⁺ concentrations.

### Example 7

### Stim2 And not Stim1 is Fully Active for Partial Store-Depleted Conditions

We further tested the hypothesis of different Ca²⁺ sensitivities of Stim1 and Stim2 by using a functional readout. Cytosolic Ca²⁺ levels were measured as a function of YFP-Stim1 or YFP-Stim2 expression for normal and reduced ER loading levels (FIG. 4D-E). Reduced ER Ca²⁺ levels were generated by exposing cells to ˜0.1 mM extracellular Ca²⁺ for 5 hours. We used mutant STIM proteins that cannot bind Ca²⁺ (constitutively active EF-hand mutants) and functionally compared them to the wildtype proteins that can bind Ca²⁺. Expression of increasing levels of EF-hand mutants of Stim1 or Stim2 triggered relative Ca²⁺ increases that were, as expected, insensitive to the ER loading levels (FIG. 4D-E). For the condition of normal ER Ca²⁺, wildtype Stim1 and Stim2 were less potent in raising Ca²⁺ than their EF-hand mutant counterparts (FIG. 4D-E). This is consistent with the ability of both isoforms to be inhibited by binding Ca²⁺ at normal ER Ca²⁺ levels. A clear difference between Stim1 and Stim2 function can be seen for reduced ER Ca²⁺ levels. Stim2 was as potent as its EF-hand mutant in raising Ca²⁺, indicating that Ca²⁺ had completely dissociated from Stim2 for the reduced ER Ca²⁺ condition (FIG. 4D). Stim1, by contrast, was less potent than the Stim1 EF-hand mutant, suggesting that it was still partially inhibited by Ca²⁺ (FIG. 4E). This functional comparison provides additional evidence that Stim2 activity becomes partially suppressed by Ca²⁺ only for high, near basal, ER-Ca²⁺ concentrations, while the activity of Stim1 is suppressed at a much lower ER Ca²⁺ concentration.

### Example 8

### Stim2-Regulation of Ca 2-Influx does not Require Stim1

Since Stim1 and Stim2 have been shown to be able to form hetero-oligomers (Dziadek and Johnstone, 2007; Stathopulos, 2006), it is plausible that they function as a complex and that Stim2 requires Stim1 to signal to Orai1 channels for both B-SOC or R-SOC type Ca²⁺ influx. We used over-expression of Stim2 combined with Stim1 knockdown to test such a possible co-regulation. FIG. 5A shows that Stim1 knockdown did not affect the increase in basal Ca²⁺ resulting from Stim2 expression. Furthermore, Stim1 knockdown also did not interfere with the ability of YFP-Stim2 expression to enhance store-depletion-triggered Ca²⁺-influx (FIG. 5B). For comparison, knockdown of ORAI1 markedly suppressed the ability of expressed Stim2 to enhance store-operated Ca²⁺ influx. As a reciprocal control experiment, we also tested for a role of Stim2 in Stim1 signaling and performed an analogous experiment using YFP-Stim1 expression combined with Stim2 knockdown. We found only a small effect of Stim2 knockdown on the ability of YFP-Stim1 to trigger store-depletion triggered Ca²⁺ influx (FIG. 5C). Again, knockdown of ORAI1 markedly suppressed the ability of expressed Stim1 to enhance store-operated Ca²⁺ influx. Thus, while basal Ca²⁺ is primarily controlled by Stim2, influx under store-depleted conditions can be independently regulated by Stim1 or Stim2 and both signaling proteins function upstream of Orai1 (FIG. 5D).

### REFERENCES

Berridge, M. J., Bootman, M. D., and Roderick, H. L. (2003). Ca²⁺ signalling: dynamics, homeostasis and remodelling. Nat Rev Mol Cell Biol. 4, 517-29.
Bezprozvanny, I. and Hayden, M. R. (2004). Deranged neuronal Ca²⁺ signaling and Huntington disease. Biochem Biophys Res Commun. 322, 1310-7.
Brini, M., Pinton, P., Pozzan, T., and Rizzuto, R. (1999). Targeted recombinant aequorins: tools for monitoring [Ca²⁺] in the various compartments of a living cell. Microsc Res Tech. 46, 380-9.
Camello, C., Lomax, R., Petersen, O. H., and Tepikin, A. V. (2002). Calcium leak from intracellular stores—the enigma of calcium signalling. Cell Calcium. 32, 355-61.
Campanella, M., Pinton, P., and Rizzuto, R. (2004). Mitochondrial Ca²⁺ homeostasis in health and disease. Biol Res. 37:653-60.
Dziadek, M. A. and Johnstone, L. S. (2007). Biochemical properties and cellular localisation of STIM proteins. Cell Ca²⁺. (epub March 21).
Feske, S., Gwack, Y., Prakriya, M., Srikanth, S., Puppel, S. H., Tanasa, B., Hogan, P. G., Lewis, R. S., Daly, M., and Rao, A. (2006). A mutation in ORAI1 causes immune deficiency by abrogating CRAC channel function. Nature. 441, 179-85.
Gallo, E. M., Cante-Barrett, K., and Crabtree, G. R. (2006). Lymphocyte Ca²⁺ signaling from membrane to nucleus. Nat Immunol. 7, 25-32.
Grynkiewicz, G., Poenie, M., and Tsien, R. Y. (1985). A new generation of Ca²⁺ indicators with greatly improved fluorescence properties. J Biol Chem. 260, 3440-50.
Guerini, D., Coletto, L., and Carafoli, E. (2005). Exporting Ca²⁺ from cells. Cell Ca²⁺. 38, 281-9.
Kusters, J. M., Demison, M. M., van Meerwijk, W. P., Ypey, D. L., Theuvenet, A. P., and Gielen, C. C. (2005). Stabilizing role of calcium store-dependent plasma membrane calcium channels in action-potential firing and intracellular calcium oscillations. Biophys J. 89, 3741-56.
Liou, J., Fivaz, M., Inoue, T., and Meyer T. (2007). Live-cell imaging reveals sequential oligomerization and local plasma membrane targeting of stromal interaction molecule 1 after Ca²⁺ store depletion. Proc Natl Acad Sci USA. 104, 9301-6.
Liou, J., Kim. M. L., Heo, W. D., Jones, J. T., Myers, J. W., Ferrell, J. E. Jr., and Meyer, T. (2005). STIM is a Ca²⁺ sensor essential for Ca²⁺-store-depletiontriggered Ca²⁺ influx. Curr Biol. 15, 1235-41.
Luik, R. M, Wu, M. M., Buchanan, J., and Lewis R S. (2006). The elementary unit of store-operated Ca²⁺ entry: local activation of CRAC channels by Stim1 at ERplasma membrane junctions. J Cell Biol. 174, 815-25.
Mattson, M. P. (2007). Ca²⁺ and neurodegeneration. Aging Cell. 6, 337-50. Mercer, J. C., Dehaven, W. I., Smyth, J. T., Wedel, B., Boyles, R. R., Bird, G. S., and Putney, J. W. Jr. (2006). Large store-operated Ca²⁺ selective currents due to co-expression of ORAI1 or Orai2 with the intracellular Ca²⁺ sensor, Stim1. J Biol Chem. 281, 24979-90.
Myers, J. W., Jones, J. T., Meyer, T., and Ferrell, J. E. Jr. (2003). Recombinant Dicer efficiently converts large dsRNAs into siRNAs suitable for gene silencing. Nat Biotechnol. 21, 324-8.
Peinelt, C., Vig, M., Koomoa, D. L., Beck, A., Nadler, M. J., Koblan-Huberson, M., L is, A., Fleig, A., Penner, R., and Kinet, J. P. (2006). Amplification of CRAC current by Stim1 and CRACM1 (ORAI1). Nat Cell Biol. 8, 771-3.
Periasamy, M., and Kalyanasundaram, A. (2007). SERCA pump isoforms: their role in calcium transport and disease. Muscle Nerve. 35, 430-42.
Philipson, K. D., Nicoll, D. A., Ottolia M, Quednau B D, Reuter H, John S, Qiu Z. (2002). The Na+/Ca²⁺ exchange molecule: an overview. Ann NY Acad Sci. 976, 1-10.
Pinton, P., and Rizzuto, R. (2006). Bcl-2 and Ca²⁺ homeostasis in the endoplasmic reticulum. Cell Death Differ. 13, 1409-18.
Raza, M., Deshpande, L. S., Blair, R. E., Carter, D. S., Sombati, S., and Delorenzo, R. J. (2007). Aging is associated with elevated intracellular Ca²⁺ levels and altered Ca²⁺ homeostatic mechanisms in hippocampal neurons. Neurosci Lett. 418, 77-81.
Roos, J., DiGregorio, P. J., Yeromin, A. V., Ohlsen, K., Lioudyno, M., Zhang, S., Safrina, O., Kozak, J. A., Wagner, S. L., Cahalan, M. D., Velicelebi, G., and Stauderman, K. A. (2005). Stim1, an essential and conserved component of store-operated Ca²⁺ channel function. J Cell Biol. 169, 435-45.
Schulman, I. H, Zachariah, M., and Raij, L. (2005). Ca²⁺ channel blockers, endothelial dysfunction, and combination therapy. Clin Exp Res. 17, 40-5.
Soboloff, J., Spassova, M. A., Hewavitharana, T., He, L. P., Xu, W., Johnstone, L. S., Dziadek, M. A., and Gill, D. L. (2006a). Stim2 is an inhibitor of Stim1-mediated store-operated Ca²⁺ Entry. Curr Biol. 16, 1465-70.
Soboloff, J., Spassova, M. A., Tang, X. D., Hewavitharana, T., Xu, W., and Gill, D. L. (2006b). ORAI1 and STIM reconstitute store-operated Ca²⁺ channel function. J Biol Chem. 281, 20661-5.
Spira, M. E., Oren, R., Dormann, A., Ilouz, N., and Lev, S. (2001). Calcium, protease activation, and cytoskeleton remodeling underlie growth cone formation and neuronal regeneration. Cell Mol Neurobiol. 21, 591-604.
Stathopulos, P. B., Li, G. Y., Plevin, M. J., Ames, J. B., and Ikura, M. (2006). Stored Ca²⁺ depletion-induced oligomerization of stromal interaction molecule 1 (Stim1) via the EF-SAM region: An initiation mechanism for capacitive Ca²⁺ entry. J Biol Chem. 281, 35855-62.
Strehler, E. E., Caride, A. J., Filoteo, A. G., Xiong, Y., Penniston, J. T., and Enyedi, A. (2007). Plasma membrane Ca²⁺ ATPases as dynamic regulators of cellular calcium handling. Ann NY Acad Sci. 1099, 226-36.
Stutzmann, G. E. (2005). Ca²⁺ dysregulation, IP3 signaling, and Alzheimer's disease. Neuroscientist. 11, 110-5.
Ter Keurs, H. E. and Boyden, P. A. (2007). Ca²⁺ and arrhythmogenesis. Physiol Rev. 87, 457-506.
Thastrup, O., Cullen, P. J., Drobak, B. K., Hanley, M. R., and Dawson, A. P. (1990). Thapsigargin, a tumor promoter, discharges intracellular Ca²⁺ stores by specific inhibition of the endoplasmic reticulum Ca2(+)-ATPase. Proc Natl Acad Sci USA. 87, 2466-70.
Thebault, S., Hoenderop, J. G., and Bindels, R. J. Epithelial Ca²⁺ and Mg²⁺ channels in kidney disease. (2006). Adv Chronic Kidney Dis. 13, 110-7.
Treves, S., Anderson, A. A., Ducreux, S., Divet, A., Bleunven, C., Grasso, C., Paesante, S., and Zorzato, F. (2005). Ryanodine receptor 1 mutations, dysregulation of Ca²⁺ homeostasis and neuromuscular disorders. Neuromuscul Disord. 15, 577-87.
Tu, H., Nelson, O., Bezprozvanny, A., Wang, Z., Lee, S. F., Hao, Y. H., Serneels, L., De Strooper, B., Yu, G., and Bezprozvanny, I. (2006). Presenilins form ER Ca²⁺ leak channels, a function disrupted by familial Alzheimer's disease-linked mutations. Cell. 126, 981-93.
Wu, M. M., Buchanan, J., Luik, R. M., and Lewis, R. S. (2006). Ca²⁺ store depletion causes Stim1 to accumulate in ER regions closely associated with the plasma membrane. J Cell Biol. 174, 803-13.
Yeromin, A. V., Zhang, S. L., Jiang, W., Yu, Y., Safrina, O., and Cahalan, M. D. (2006). Molecular identification of the CRAC channel by altered ion selectivity in a mutant of Orai. Nature. 443, 226-9.
Yu, R. and Hinkle, P. M. (2000). Rapid turnover of calcium in the endoplasmic reticulum during signaling. Studies with cameleon calcium indicators. J Biol Chem. 275, 23648-53.
Zatti, G., et al. (2006). Presenilin mutations linked to familial Alzheimer's disease reduce endoplasmic reticulum and Golgi apparatus Ca²⁺ levels. Cell Ca²⁺. 39, 539-50.
Zhang, K. and Kaufman, R. J. (2006). The unfolded protein response: a stress signaling pathway critical for health and disease. Neurology. 66, S102-9.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions.

Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

That which is claimed is:

1. A method of assessing the basal calcium level state in a cell, the method comprising:
providing a cell comprising a STIM-2 polypeptide;
detecting a distribution pattern of said STIM-2 polypeptide in said cell; and
assessing the basal calcium level state in said cell based on said distribution pattern.

2. The method of claim 1, wherein the STIM-2 polypeptide comprises a detectible domain.

3. The method of claim 2, wherein the detectable domain is a fluorescent polypeptide.

4. The method of claim 1, wherein a punctate STIM-2 distribution pattern is indicative of depleted basal intracellular calcium levels.

5. The method of claim 1, wherein a diffuse STIM-2 distribution pattern is indicative of levels of basal intracellular calcium that are not depleted.

6. A method of identifying candidate agents that modulate basal calcium levels in a cell, said method comprising:
contacting said cell with a candidate agent;
assessing basal calcium level changes in said cell as a result of said contacting; and
identifying said candidate agent as a modulator of basal calcium levels in said cell based on said assessing.

7. The method of claim 6, wherein said cells are cultured in calcium sensitizing conditions prior to said contacting.

8. The method of claim 7, wherein said calcium sensitizing conditions comprises culturing said cell in high levels of extracellular calcium, and wherein said agent is identified as modulator of basal calcium levels when the assessed basal calcium level increases after said contacting step.

9. The method of claim 7, wherein said sensitizing condition comprises culturing said cell in low levels of extracellular calcium, and wherein said agent is identified as modulator of basal calcium levels when the assessed basal calcium level decreases after said contacting step.

10. The method of claim 7, wherein said culturing step comprises culturing said cell in a first and a second distinct sensitizing condition, wherein said first sensitizing condition comprises culturing said cell in high levels of extracellular calcium and said second sensitizing condition comprises culturing said cell under low levels of extracellular calcium.

11. The method of claim 6, wherein said cells express a detectible STIM protein and wherein said assessing comprises detecting the distribution of said detectible STIM protein.

12. The method of claim 6, wherein said candidate agent is selected from one or more of: proteins, oligopeptides, small molecules, polysaccharides, polynucleotides, and RNAi agents.

13. A method of modulating basal calcium levels in a cell, said method comprising:
contacting said cell with an agent that modulates STIM-2 activity in said cell, wherein said basal calcium levels in said cell are modulated.

14. The method of claim 13, wherein said modulating said STIM-2 activity includes one or more of: modulating STIM-2 calcium binding, modulating STIM-2 aggregation, modulating STIM-2 expression level, and modulating STIM-2-mediated calcium transport.

15. The method of claim 14, wherein said agent increases said STIM-2 activity, thereby increasing basal calcium levels in said cell.

16. The method of claim 14, wherein said agent decreases said STIM-2 activity, thereby decreasing basal calcium levels in said cell.

17. A method of treating a subject having a condition associated with dysregulated cellular basal calcium levels, said method comprising:
administering to said subject an effective amount of an agent that modulates cellular STIM-2 activity, wherein said condition associated with dysregulated cellular basal calcium levels is treated in said subject.

18. The method of claim 17, wherein said modulating said STIM-2 activity includes one or more of: modulating STIM-2 calcium binding, modulating STIM-2 aggregation, modulating STIM-2 expression level, and modulating STIM-2-mediated calcium transport.

19. The method of claim 17, wherein said condition is characterized by low basal calcium levels and said agent increases said cellular STIM-2 activity.

20. The method of claim 17, wherein said condition is characterized by high basal calcium levels and said agent decreases said cellular STIM-2 activity.
